(19) United States
(12) Patent Application Publication
(10) Pub. No.: US 2019/0201501 A1
(43) Pub. Date: Jul. 4, 2019

(54) COMPOSITIONS AND METHODS FOR CELL TARGETED HPV TREATMENT

(71) Applicant: Agenovir Corporation, South San Francisco, CA (US)

(72) Inventors: Stephen R. Quake, Stanford, CA (US); Jianbin Wang, South San Francisco, CA (US)

(21) Appl. No.: 16/224,591

(22) Filed: Dec. 18, 2018

Related U.S. Application Data
(63) Continuation of application No. 15/166,936, filed on May 27, 2016, now abandoned.
(60) Provisional application No. 62/168,188, filed on May 29, 2015.

(51) Int. Cl. A61K 38/46; C12N 9/22; A61K 9/127; A61K 31/7105

(52) U.S. Cl. CPC A61K 38/465; C12N 9/22; A61K 9/127; C07K 2319/01; C12Y 301/00; C07K 2319/85; A61K 31/7105

(57) ABSTRACT

The invention provides compositions and methods for treating human papillomavirus (HPV) infections using a targetable nuclease, which compositions and methods can be used to selectively target the HPV genome or selectively express the targetable nuclease within cells that infected by HPV. By selectively targeting cells infected by HPV, the HPV genome within infected cells, or both, the nuclease is able to cleave the HPV genome thereby inactivating it and rendering it inoperable, interfering with the virus's ability to propagate even where the virus is in a latent stage of infection. Since latent HPV can be cleaved and eradicated from the host cells, compositions and methods of the invention may be used to treat HPV infections and potentially prevent many of the adverse health consequences associated with the papillomavirus.

### CROSS-REFERENCE TO RELATED APPLICATION

This application claims the benefit and priority of U.S. Provisional Patent Application No. 62/168,188, filed May 29, 2015, the contents of which are incorporated by reference.

### FIELD OF THE INVENTION

The invention generally relates to compositions and methods for selectively treating viral infections using a guided nuclease system.

### BACKGROUND

Human papillomavirus, or HPV, is a virus that infects many people. In fact, more than 75% of women and men will be infected at some point in life. While most HPV infections are subclinical and will cause no physical symptoms, in some people infections may cause growths known as papillomas, and may even cause cancers of the cervix, vulva, vagina, penis, oropharynx and anus. In particular, HPV16 and HPV18 are known to cause around 70% of cervical cancer cases.

High-risk oncogenic HPV types are able to integrate into the host DNA and express genes such as HPV E6 and E7. It is thought that the E6 and E7 oncoproteins inactivate p53 and pRB tumor suppressors, implicating HPV in the development of cancer. HPV also undergoes a latency stage in which it has the ability to lie dormant within a cell indefinitely and not be fully eradicated even after treatment. The result is that the virus can reactivate long after an infection and begin replicating and expressing its genes.

### SUMMARY

The invention provides compositions and methods for treating viral infections using a targetable nuclease, which compositions and methods can be used to selectively target the HPV genome or selectively express the targetable nuclease within cells that are infected by HPV or cells of a certain type. By selectively targeting cells of a certain type of those infected by HPV, targeting the HPV genome within infected cells, or a combination thereof, the nuclease is able to cleave the HPV genome thereby inactivating it and rendering it inoperable, interfering with the virus's ability to propagate even where the virus is in a latent stage of infection. Targeting the infected cells selectively can be done using a cell-type specific promoter, e.g., for keratinocytes, where such cells are the infected cells. Due to the targetable nature of the nuclease, it can be used to cleave the HPV genome without interfering with normal function of the host human genome. Targeting the viral nucleic acid can be done using a sequence-specific moiety such as a guide RNA that targets viral genomic material for destruction by the nuclease and does not target the host cell genome. In some embodiments, a CRISPR/Cas9 nuclease and guide RNA (gRNA) that together target and selectively edit or destroy viral genomic material is used. The gRNA targets Cas9 to a specific portion of the HPV genome. Since latent HPV can be cleaved and eradicated from the host cells, compositions and methods of the invention may be used to treat HPV infections and potentially prevent many of the adverse health consequences associated with the papillomavirus.

Aspects of the invention provide a composition that includes a ribonucleoprotein (RNP) comprising an RNA-guided nuclease and an RNA with a portion complementary to a target site within a viral nucleic acid of the virus. The RNP is preferably extra-cellular in that it exists in active form in solution outside of any cell.

In certain embodiments, the RNA-guided nuclease is selected from the group consisting of a CRISPR-associated protein and Cpf1. The composition may include a liposome enveloping the RNP. The virus may be, e.g., human papillomavirus (HPV). In the embodiments, the target site may lie within an E6 or E7 gene of a genome of the HPV. Optionally, the RNA-guided nuclease may include a nuclear localization signal. The composition may include at least a second RNP that itself includes a second RNA-guided nuclease and a second RNA. The second RNA includes a second portion complementary to a second target site within the viral nucleic acid, and the second target site lies within the E6 or E7 gene and is not the same as the target site. The composition may include a liposome enveloping the RNP and a second liposome enveloping the second RNP. In one of the certain embodiments, the RNA-guided nuclease is Cas9.

In some embodiments, the composition includes a plurality of RNPs that when delivered to cells infected with the virus cleave the viral nucleic acid in a plurality of locations. The composition may further include a plurality of liposomes enveloping the plurality of RNPs.

In any of the embodiments, it may be preferable that the portion complementary to the target site has no match >60% within a human genome.

Aspects of the invention provide a method of removing foreign nucleic acid from cells. The method includes delivering to cells or tissue in vitro a composition that includes an extra-cellular RNP according to any of the embodiments described above and cleaving viral nucleic acid with the RNP.

In certain aspects, the invention provides a composition for treating a human papillomavirus (HPV) infection. The composition includes a vector encoding a targetable nuclease and one or more targeting sequence that targets the nuclease to an HPV genome. The vector may include an inducible promoter that promotes expression of the targetable nuclease and the targeting sequence within a keratinocyte. For example, the inducible promoter could be a promoter-enhancer cassette that selectively favors expression of the targetable nuclease and the targeting sequence within the keratinocyte over other types of host cells. The targetable nuclease may be a zinc-finger nuclease, a transcription activator-like effector nuclease, a meganuclease, or any other suitable targetable nuclease.

In a preferred embodiment, the nuclease is a Cas9 endonuclease and the targeting sequence or sequences comprise a guide RNA. The targeting sequence or sequences may target the nuclease to cleave a specific gene within the HPV genome, such as the E6 gene, the E7 gene, others, or a combination thereof. Preferably the targeting sequence is a guide RNA and has no match >60% within a human genome.

The composition may be packaged for delivery to a human patient, e.g., in or with a subdermal or intravenous delivery system. The vector could include using a retrovirus, lentivirus, adenovirus, herpesvirus, poxvirus, alphavirus, vaccinia virus, adeno-associated viruses, a plasmid, a nanoparticle, a cationic lipid, a cationic polymer, metallic nanoparticle, a nanorod, a liposome, microbubbles, a cell-penetrating peptide, or a liposphere.

Aspects of the invention provide a method for treating a human papillomavirus (HPV) infection. The method includes introducing into a host cell a targetable nuclease and a targeting sequence that targets the nuclease to an HPV genome. The HPV genome is cleaved with the nuclease within the host cell without interfering with genes on a host genome. In some embodiments, the targetable nuclease and the targeting sequence are introduced directly—e.g., as a protein and one or more guide RNA—to a patient (e.g., by injection or intravenously). In certain embodiments, the targetable nuclease and the targeting sequence are introduced using a vector that encodes the targetable nuclease and the targeting sequence. Methods of the invention also include introducing the nuclease and targeting sequence in vitro, e.g., for a cellular assay.

In a preferred embodiment, the host cell is a keratinocyte and the vector includes a feature that promotes expression of the targetable nuclease and the targeting sequence within the keratinocyte. The feature that promotes expression may be a promoter-enhancer cassette that selectively favors expression of the targetable nuclease and the targeting sequence within the keratinocyte over other types of host cells. Any suitable nuclease such as a zinc-finger nuclease, a transcription activator-like effector nuclease, or a meganuclease could be used. In the preferred embodiment of the method, the nuclease is Cas9 endonuclease and the targeting sequence comprises a guide RNA. The targeting sequence or sequences may target the nuclease to cleave a specific gene within the HPV genome, such as the E6 gene, the E7 gene, others, or a combination thereof and the targeting sequence or sequences are guide RNAs that have no match >70% within a human genome.

Aspects of the invention provide a composition that comprises: a ribonucleoprotein (RNP) comprising an RNA-guided nuclease; and an RNA with a portion complementary to a target site within a viral nucleic acid of the virus. Preferably the portion complementary to the target site has no match >60% within a human genome. The RNA-guided nuclease may be selected from the group consisting of a CRISPR-associated protein and Cpf1. In certain embodiments, the RNA-guided nuclease is Cas9. The composition may further include a liposome enveloping the RNP. Such a composition may be used in a method of removing foreign nucleic acid (e.g., viral) from cells, the method comprising delivering to cells or tissue in vitro the and cleaving the foreign nucleic acid with the RNP.

The composition may include a plurality of RNPs that when delivered to cells infected with the virus cleave the viral nucleic acid in a plurality of locations, and may even further include a plurality of liposomes enveloping the plurality of RNPs.

In some embodiments, the virus is human papillomavirus (HPV). The target site may lie within an E6 or E7 gene of a genome of the HPV. The composition may further comprise at least a second RNP, the second RNP comprising a second RNA-guided nuclease and a second RNA. The second RNA includes a second portion complementary to a second target site within the viral nucleic acid, and the second target site lies within the E6 or E7 gene and is not the same as the target site. The composition may include a liposome enveloping the RNP and a second liposome may enveloping the second RNP.

### BRIEF DESCRIPTION OF THE DRAWINGS

FIG. 1 diagrams a method for treating an HPV infection.

FIG. 2 shows targets for a targetable nuclease.

FIG. 3 gives results from targeting an HPV genome using a targetable nuclease.

FIG. 4 shows a composition for targeting an HPV genome.

FIG. 5 shows the EGFP marker fused after the Cas9 protein, allowing selection of Cas9-positive cells.

FIG. 6 shows that including an ori-P in the plasmid promoted active plasmid replication inside the cells, which increased the transfection efficiency to >60%.

FIG. 7 is a diagram of an EBV genome, with structure-, transformation-, and latency-related targets called out.

FIG. 8 shows the genome context around guide RNA sgEBV2 and PCR primer locations.

FIG. 9 shows the large deletion induced by sgEBV2 (lanes 1-3 are before, 5 days after, and 7 days after sgEBV2 treatment, respectively).

FIG. 10 shows the genome context around guide RNA sgEBV3/4/5 and PCR primer locations.

FIG. 11 shows the large deletions induced by sgEBV3/5 and sgEBV4/5. Lane 1 and 2 are 3F/5R PCR amplicons before and 8 days after sgEBV3/5 treatment. Lane 3 and 4 are 4F/5R PCR amplicons before and 8 days after sgEBV4/5 treatment.

FIG. 12 shows that Sanger sequencing confirmed genome cleavage and repair ligation 8 days after sgEBV3/5.

FIG. 13 shows that Sanger sequencing confirmed genome cleavage and repair ligation 8 days after sgEBV4/5.

FIG. 14 shows relative cell proliferation after targeting various combinations of regions in an EBV genome with guide RNAs.

FIG. 15 gives flow cytometry scattering signals from before sgEBV1-7 treatments.

FIG. 16 gives flow cytometry scattering signals from 5 days after sgEBV1-7 treatments FIG. 17 gives flow cytometry scattering signals from 8 days after sgEBV1-7 treatments.

FIG. 18 shows Annexin V Alexa647 and DAPI staining results before sgEBV1-7 treatments.

FIG. 19 shows Annexin V Alexa647 and DAPI staining results 5 days after sgEBV1-7 treatments.

FIG. 20 shows Annexin V Alexa647 and DAPI staining results 8 days after sgEBV1-7 treatments.

FIGS. 21 and 22 show microscopy revealed apoptotic cell morphology after sgEBV1-7 treatment.

FIG. 23 shows nuclear morphology before sgEBV1-7 treatment.

FIGS. 24-26 show nuclear morphology after sgEBV1-7 treatment.

FIG. 27 shows EBV load after different CRISPR treatments by digital PCR. Cas9 and Cas9-oriP had two replicates, and sgEBV1-7 had 5 replicates.

FIG. 28 shows a single Raji cell as captured on a microfluidic chip.

FIG. 29 shows a single sgEBV1-7 treated cell as captured on the chip.

FIG. 30 is a histogram of EBV quantitative PCR Ct values from single cells before treatment.

FIG. 31 is a histogram of EBV quantitative PCR Ct values from single live cells 7 days after sgEBV1-7 treatment.

FIG. 32 represents SURVEYOR assay of EBV CRISPR (lanes numbered from left to right: Lane 1: NEB 100 bp ladder; Lane 2: sgEBV1 control; Lane 3: sgEBV1; Lane 4: sgEBV5 control; Lane 5: sgEBV5; Lane 6: sgEBV7 control; Lane 7: sgEBV7; Lane 8: sgEBV4).

FIG. 33 shows that the CRISPR treatments were not cytotoxic to the EBV-negative Burkitt's lymphoma cell line DG-75

FIG. 34 shows that the CRISPR treatments were not cytotoxic to primary human lung fibroblasts IMR90.

FIG. 35 shows ZFN being used to cut viral nucleic acid.

FIG. 36 diagrams a method for treating a cell to remove foreign nucleic acid.

FIG. 37 diagrams an experimental design to show that EBV-specific CRISPR/Cas9 RNP specifically kills EBV+B lymphoma cancer cells.

FIG. 38 shows EBV+ cancer cell survival for 6 days post-treatment.

FIG. 39 shows the percent of each cell population at day 6 post-treatment.

FIG. 40 shows the percent cell survival for 3 days after treatment.

FIG. 41 shows where the selected guide RNAs map to a genome.

FIG. 42 shows the percent survival after treatment with Cas9.

FIG. 43 shows the HPV-specific CRISPR/Cas9 RNP dose response.

FIG. 44 gives the HPV-specific CRISPR/Cas9 RNP time-course.

FIG. 45 is a gel showing that CRISPR/Cas9 RNP Enhances DNA cleavage.

FIG. 46 shows that RNP has decreased cytotoxicity relative to pDNA.

FIG. 47 shows that HPV+ cancer cell survival is lower for RNP versus pDNA.

FIG. 48 shows that a combination of HPV-Specific CRISPR RNPs improves HPV+ cancer cell killing.

FIG. 49 shows the primer design for killing HPV+ cancer cells.

FIG. 50 shows a process for monitoring cell survival.

FIG. 51 shows that HPV-16 specific CRISPR/Cas9 pDNA kills HPV-16 positive cancer cells.

FIG. 52 illustrated delivery of Cas9 RNP via liposome.

FIG. 53 shows that HPV-Specific CRISPR/Cas9 RNP formulated into a liposome inhibits HPV+ cancer cells.

### DETAILED DESCRIPTION

The invention generally relates to compositions and methods for selectively treating viral infections using a guided nuclease system with particular application to HPV infections in keratinocytes. Methods of the invention are used to incapacitate or disrupt viral nucleic acid within a cell through nuclease activity such as single- or double-stranded breaks, cleavage, digestion, or editing. Methods of the invention may be used for systematically causing large or repeated deletions in the genome, reducing the probability of reconstructing the full genome.

Compositions and methods of the invention are provided to treat HPV infections as well as symptoms and consequences of HPV infection. HPV establishes productive infections only in keratinocytes of the skin or mucous membranes. The invention provides compositions and methods for nuclease-based antiviral therapy against HPV infection with applicability against latent HPV infection.

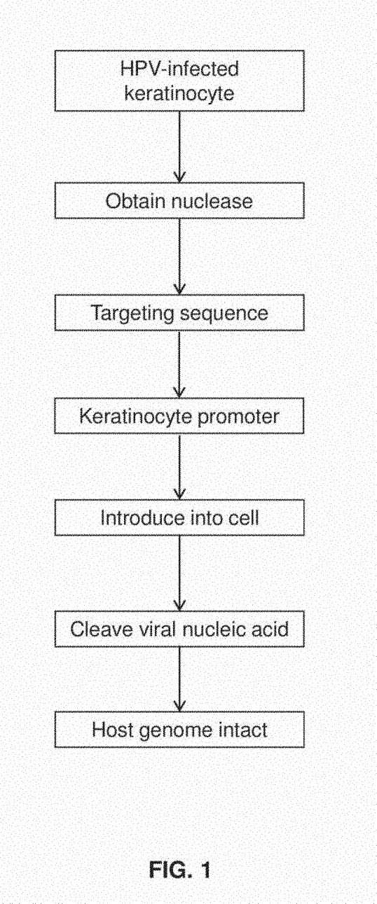

FIG. 1 diagrams a method of targeting an HPV infection. Preferably a composition of the invention is delivered to a keratinocyte. It is understood that clinical significant HPV infections affect keratinocytes. An HPV infected keratinocyte in vivo may be treated according to methods of the invention. The method includes obtaining a targetable nuclease (e.g., as a protein or a gene for a nuclease). Any suitable nuclease can be used such as ZFN, TALENs, or meganucleases. In a preferred embodiment, the nuclease is Cas9. A sequence is provided that targets the nuclease to specific targets on the HPV genome. The sequence may be in the form of DNA that is complementary to guide-RNA, which sequence will be transcribed within the keratinocyte to provide the final gRNA. The nuclease gene and encoded gRNAs may be provided in a DNA vector, such as a plasmid or an adenovirus based vector, and the vector may further optionally include a keratinocyte-specific inducible promoter. That composition is then introduced into the HPV-infected cells. Any suitable transfection or delivery method may be used. Once in the cell, the genes are expressed and the Cas9 enzyme uses the gRNA to target, and cleave, the HPV genome. Since the gRNA is specific to the HPV genome with no match to the human genome according to methods and criteria described herein, the method leaves the host genome intact and does not interfere with normal human genetic function. Discussion of HPV may be found in Munger et al., 2004, Mechanisms of human papillomavirus-induced oncogenesis, J Virol 78(21):11451-11460 and Madkan et al., 2007, The oncogenic potential of human papillomaviruses: a review on the role of host genetics and environmental cofactors, Brit J Dermatology 157(2):228-241, the contents of each of which are incorporated by reference in their entirety for all purposes.

Compositions and methods can be used to selectively target the HPV genome or selectively express the targetable nuclease within cells that infected by HPV. In certain embodiments, methods and compositions of the invention use CRISPR guide RNA sequences targeting the HPV E6 and E7 genes. A composition of the invention, such as a DNA vector encoding cas9, may code for gRNAs that are complementary to specific targets within the HBV genome.

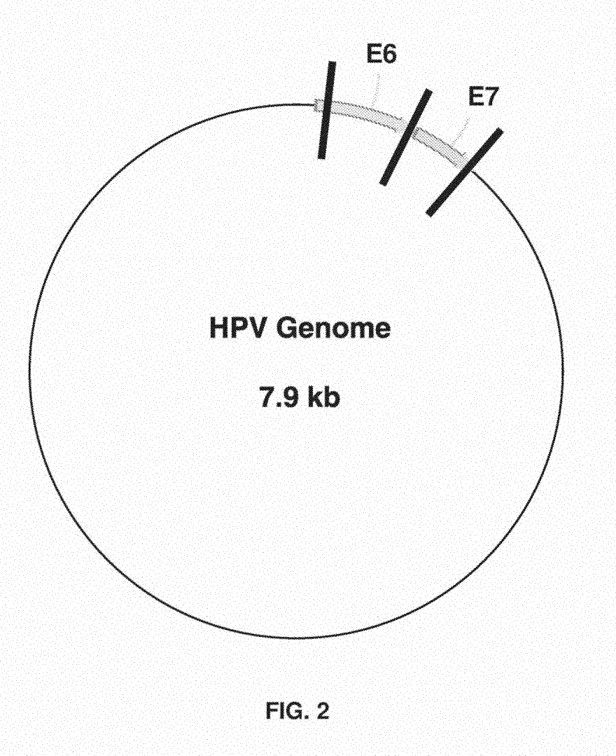

FIG. 2 shows the HPV genome and the HPV E6 and E7 genes that are targeted by CRISPR guide RNAs. Since E6 and E7 proteins may be oncogenic it may be valuable to target their respective genes for destructions by the nuclease. In a preferred embodiment, each gene is scanned for the protospacer adjacent motif (PAM) of the nuclease (e.g., 5′-NGG-3′ for Cas9). For each candidate PAM found within a gene, the 20 nt that are adjacent to the PAM are read and compared to a human genome. Where that 20-nt+PAM has no match within the human genome to a certain criteria, then that 20-nt+PAM can be used as the targeting sequence. The match criteria may be the requirement of no perfect match. In a preferred embodiment, the targeting sequence is 20-nt+PAM (e.g., 23-nt for Cas9) for which there is no 23 nt string within a human genome that matches ≥70%. In a much preferred embodiment, the targeting sequence is 20-nt+PAM for which there is no 20 nt string within the human genome that is followed by the PAM and wherein the 20 nt of human genome matches the 20 nt of targeting sequence by ≥70% (e.g., if Cas9 is the nuclease, a 20 nt string of human genome with 14 or more matching bases followed by the PAM would rule out use of a given targeting sequence).

The use of a targetable nuclease to cleave an HPV genome is shown here by an in vitro CRISPR endonuclease assay. A genetically encoded gRNA scaffold was provided for transcription by a T7 phage polymerase. T7 in vitro transcription produced the complete guide RNA with scaffold. Flanking regions of the genome targets were PCR amplified from HPV18 genomic DNA (sold under the trademark 45152D by ATCC of Manassas, Va.). Cas9 protein (from PNA Bio of Thousand Oaks, Calif.), guide RNA and target DNA were mixed and incubated for in vitro endonuclease assay. High endonuclease activities were revealed by DNA gel electrophoresis of the digested DNA.

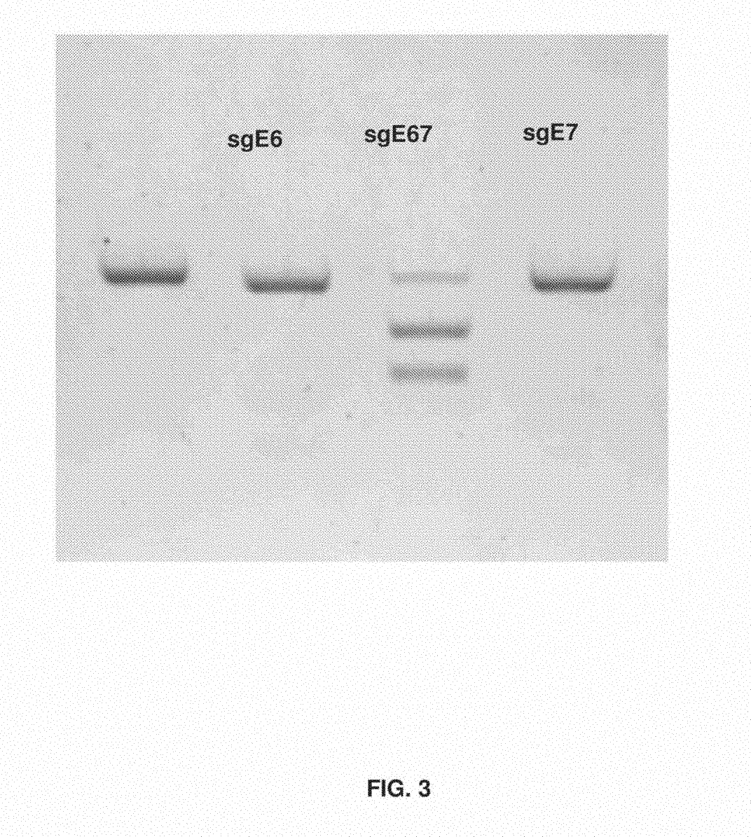

FIG. 3 gives the results of the in vitro CRISPR endonuclease assay. Four lanes show the results of PCR amplicon of the E6-E7 region, and the products of in vitro CRISPR treated amplicons. Lanes 2-4 each show difference relative to control. Lane 3 shows cleavage of the HPV genomic DNA into three fragments of distinct masses. Since the gRNA is designed to match within the E6 or E7 gene, expression of the corresponding proteins may be stopped by nuclease cleavage.

Compositions and methods can be used to selectively express the targetable nuclease within cells that infected by HPV. It is understood that HPV infects keratinocytes. See e.g., Bossens, 1992, J Gen Virol 73:3269, incorporated by reference. In certain embodiments, a nuclease is provided with a promoter-enhancer cassette to regulate expression of the nuclease in vivo or in vitro and cause the expression of the nuclease within keratinocytes.

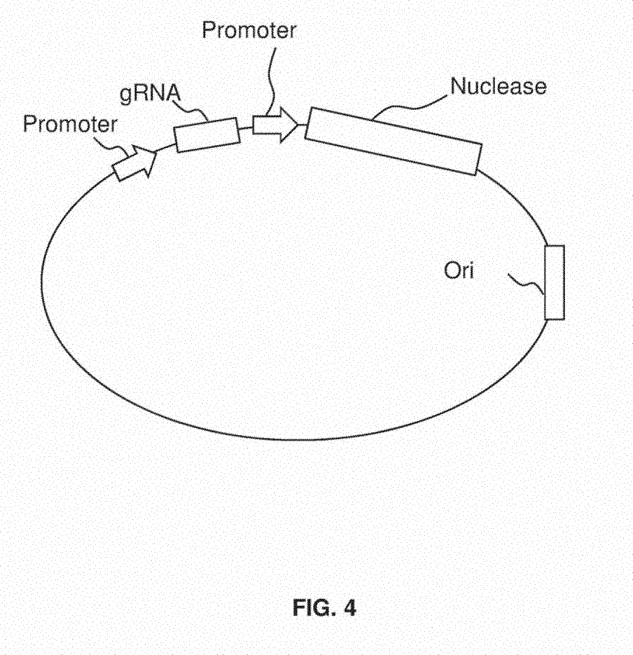

FIG. 4 shows a diagram of a composition according to certain embodiments of the invention. The composition preferably includes a DNA strand (circular or linear, here shown as circularized) that includes at least nuclease gene and at least one targeting sequence (labelled gRNA in FIG. 4). The composition may include an origin of replication such as an HPV origin. Preferably, the composition includes one or more promoters, any or all of which may be specific to keratinocytes. Any suitable promoter or enhancer may be used that results in expression within keratinocytes. For example, a nuclease may be provided within a vector (e.g., a plasmid) that includes one or more inducible promoters such as metallothionein (MT) and 1,24-vitamin D(3)(OH)(2) dehydroxylase (VDH) promoters responded to the inducing agents, Cadmium and 1,25-vitamin D(3)(OH)(2) (VitD(3)), respectively. Keratinocyte inducible promoters are discussed in Meng et al., 2002, Keratinocyte gene therapy: cytokine gene expression in local keratinocytes and in circulation by introducing cytokine genes into skin, Exp Dermatol 11(5):456-61 and additional discussion may be found in Westergaard et al., 2001, Modulation of keratinocyte gene expression and differentiation by PPAR-selective ligands and tetradecyltheioacetic acid, J Invest Dermatol 116(5):702-12, the contents of each of which are incorporated by reference. Since the inducible promoter is specific to keratinocytes and since the one or preferably the plurality of gRNAs are each specific to a portion of the HPV genome, when the composition is administered to a patient, the encoded genes will only be expressed within keratinocytes. The nuclease will be guided by the one or more gRNAs to cleave the HPV genome. Since the gRNA has no match within a human according to a specific criteria (e.g., not ≥70% match), the host genome function will be unaffected. Thus compositions and methods of the invention may be used to treat an HPV infection. Additionally, a composition of the invention may include an HPV promoter or origin of replication. Features in the HPV genome are described in Zheng & Baker, 2006, Papillomavirus genome structure, expression, and post-transcriptional regulation, Front Biosci 11:2286-2302, incorporated by reference.

i. Treating Infected Cell

FIG. 1 diagrams a method of treating a cell infected with a virus. Methods of the invention are applicable to in vivo treatment of patients and may be used to remove any viral genetic material such as genes of virus associated with a latent viral infection. Methods may be used in vitro, e.g., to prepare or treat a cell culture or cell sample. When used in vivo, the cell may be any suitable germ line or somatic cell and compositions of the invention may be delivered to specific parts of a patient's body or be delivered systemically. If delivered systemically, it may be preferable to include within compositions of the invention tissue-specific promoters. For example, if a patient has a latent viral infection that is localized to the liver, hepatic tissue-specific promotors may be included in a plasmid or viral vector that codes for a targeted nuclease.

FIG. 4 shows a composition for treating a viral infection according to certain embodiments. The composition preferably includes a vector (which may be a plasmid, linear DNA, or a viral vector) that codes for a nuclease and a targeting moiety (e.g., a gRNA) that targets the nuclease to viral nucleic acid. The composition may optionally include one or more of a promoter, replication origin, other elements, or combinations thereof as described further herein.

ii. Nuclease

Methods of the invention include using a programmable or targetable nuclease to specifically target viral nucleic acid for destruction. Any suitable targeting nuclease can be used including, for example, zinc-finger nucleases (ZFNs), transcription activator-like effector nucleases (TALENs), clustered regularly interspaced short palindromic repeat (CRISPR) nucleases, meganucleases, other endo- or exo-nucleases, or combinations thereof. See Schiffer, 2012, Targeted DNA mutagenesis for the cure of chronic viral infections, J Virol 88(17):8920-8936, incorporated by reference.

CRISPR methodologies employ a nuclease, CRISPR-associated (Cas9), that complexes with small RNAs as guides (gRNAs) to cleave DNA in a sequence-specific manner upstream of the protospacer adjacent motif (PAM) in any genomic location. CRISPR may use separate guide RNAs known as the crRNA and tracrRNA. These two separate RNAs have been combined into a single RNA to enable site-specific mammalian genome cutting through the design of a short guide RNA. Cas9 and guide RNA (gRNA) may be synthesized by known methods. Cas9/guide-RNA (gRNA) uses a non-specific DNA cleavage protein Cas9, and an RNA oligo to hybridize to target and recruit the Cas9/gRNA complex. See Chang et al., 2013, Genome editing with RNA-guided Cas9 nuclease in zebrafish embryos, Cell Res 23:465-472; Hwang et al., 2013, Efficient genome editing in zebrafish using a CRISPR-Cas system, Nat. Biotechnol 31:227-229; Xiao et al., 2013, Chromosomal deletions and inversions mediated by TALENS and CRISPR/Cas in zebrafish, Nucl Acids Res 1-11.

CRISPR (Clustered Regularly Interspaced Short Palindromic Repeats) is found in bacteria and is believed to protect the bacteria from phage infection. It has recently been used as a means to alter gene expression in eukaryotic DNA, but has not been proposed as an anti-viral therapy or more broadly as a way to disrupt genomic material. Rather, it has been used to introduce insertions or deletions as a way of increasing or decreasing transcription in the DNA of a targeted cell or population of cells. See for example, Horvath et al., Science (2010) 327:167-170; Terns et al., Current Opinion in Microbiology (2011) 14:321-327; Bhaya et al. Annu Rev Genet (2011) 45:273-297; Wiedenheft et al. Nature (2012) 482:331-338); Jinek M et al. Science (2012) 337:816-821; Cong L et al. Science (2013) 339:819-823; Jinek M et al. (2013) eLife 2:e00471; Mali P et al. (2013) Science 339:823-826; Qi L S et al. (2013) Cell 152:1173-1183; Gilbert L A et al. (2013) Cell 154:442-451; Yang H et al. (2013) Cell 154:1370-1379; and Wang H et al. (2013) Cell 153:910-918).

In an aspect of the invention, the Cas9 endonuclease causes a double strand break in at least two locations in the genome. These two double strand breaks cause a fragment of the genome to be deleted. Even if viral repair pathways anneal the two ends, there will still be a deletion in the genome. One or more deletions using the mechanism will incapacitate the viral genome. The result is that the host cell will be free of viral infection.

In embodiments of the invention, nucleases cleave the genome of the target virus. A nuclease is an enzyme capable of cleaving the phosphodiester bonds between the nucleotide subunits of nucleic acids. Endonucleases are enzymes that cleave the phosphodiester bond within a polynucleotide chain. Some, such as Deoxyribonuclease I, cut DNA relatively nonspecifically (without regard to sequence), while many, typically called restriction endonucleases or restriction enzymes, cleave only at very specific nucleotide sequences. In a preferred embodiment of the invention, the Cas9 nuclease is incorporated into the compositions and methods of the invention, however, it should be appreciated that any nuclease may be utilized.

In preferred embodiments of the invention, the Cas9 nuclease is used to cleave the genome. The Cas9 nuclease is capable of creating a double strand break in the genome. The Cas9 nuclease has two functional domains: RuvC and HNH, each cutting a different strand. When both of these domains are active, the Cas9 causes double strand breaks in the genome.

In some embodiments of the invention, insertions into the genome can be designed to cause incapacitation, or altered genomic expression. Additionally, insertions/deletions are also used to introduce a premature stop codon either by creating one at the double strand break or by shifting the reading frame to create one downstream of the double strand break. Any of these outcomes of the NHEJ repair pathway can be leveraged to disrupt the target gene. The changes introduced by the use of the CRISPR/gRNA/Cas9 system are permanent to the genome.

In some embodiments of the invention, at least one insertion is caused by the CRISPR/gRNA/Cas9 complex. In a preferred embodiment, numerous insertions are caused in the genome, thereby incapacitating the virus. In an aspect of the invention, the number of insertions lowers the probability that the genome may be repaired.

In some embodiments of the invention, at least one deletion is caused by the CRISPR/gRNA/Cas9 complex. In a preferred embodiment, numerous deletions are caused in the genome, thereby incapacitating the virus. In an aspect of the invention, the number of deletions lowers the probability that the genome may be repaired. In a highly-preferred embodiment, the CRISPR/Cas9/gRNA system of the invention causes significant genomic disruption, resulting in effective destruction of the viral genome, while leaving the host genome intact.

TALENs uses a nonspecific DNA-cleaving nuclease fused to a DNA-binding domain that can be to target essentially any sequence. For TALEN technology, target sites are identified and expression vectors are made. Linearized expression vectors (e.g., by Notl) may be used as template for mRNA synthesis. A commercially available kit may be use such as the mMESSAGE mMACHINE SP6 transcription kit from Life Technologies (Carlsbad, Calif.). See Joung & Sander, 2013, TALENs: a widely applicable technology for targeted genome editing, Nat Rev Mol Cell Bio 14:49-55.

TALENs and CRISPR methods provide one-to-one relationship to the target sites, i.e. one unit of the tandem repeat in the TALE domain recognizes one nucleotide in the target site, and the crRNA, gRNA, or sgRNA of CRISPR/Cas system hybridizes to the complementary sequence in the DNA target. Methods can include using a pair of TALENs or a Cas9 protein with one gRNA to generate double-strand breaks in the target. The breaks are then repaired via non-homologous end-joining or homologous recombination (HR).

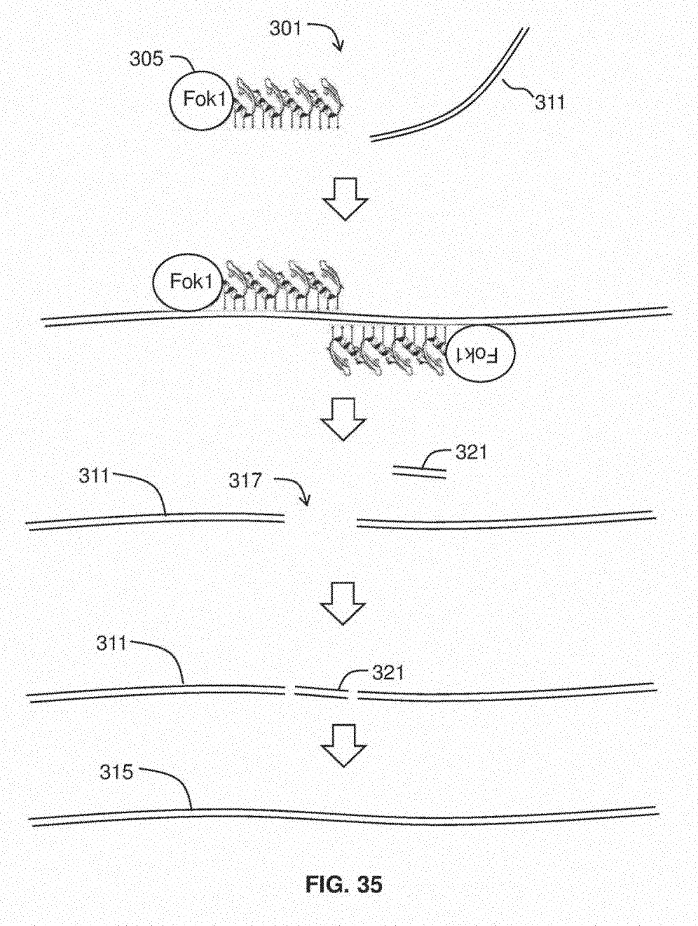

FIG. 35 shows ZFN being used to cut viral nucleic acid. Briefly, the ZFN method includes introducing into the infected host cell at least one vector (e.g., RNA molecule) encoding a targeted ZFN 305 and, optionally, at least one accessory polynucleotide. See, e.g., U.S. Pub. 2011/0023144 to Weinstein, incorporated by reference The cell includes target sequence 311. The cell is incubated to allow expression of the ZFN 305, wherein a double-stranded break 317 is introduced into the targeted chromosomal sequence 311 by the ZFN 305. In some embodiments, a donor polynucleotide or exchange polynucleotide 321 is introduced. Swapping a portion of the viral nucleic acid with irrelevant sequence can fully interfere transcription or replication of the viral nucleic acid. Target DNA 311 along with exchange polynucleotide 321 may be repaired by an error-prone non-homologous end-joining DNA repair process or a homology-directed DNA repair process.

Typically, a ZFN comprises a DNA binding domain (i.e., zinc finger) and a cleavage domain (i.e., nuclease) and this gene may be introduced as mRNA (e.g., 5′ capped, polyadenylated, or both). Zinc finger binding domains may be engineered to recognize and bind to any nucleic acid sequence of choice. See, e.g., Qu et al., 2013, Zinc-finger-nucleases mediate specific and efficient excision of HIV-1 proviral DAN from infected and latently infected human T cells, Nucl Ac Res 41(16):7771-7782, incorporated by reference. An engineered zinc finger binding domain may have a novel binding specificity compared to a naturally-occurring zinc finger protein. Engineering methods include, but are not limited to, rational design and various types of selection. A zinc finger binding domain may be designed to recognize a target DNA sequence via zinc finger recognition regions (i.e., zinc fingers). See for example, U.S. Pat. Nos. 6,607,882; 6,534,261 and 6,453,242, incorporated by reference. Exemplary methods of selecting a zinc finger recognition region may include phage display and two-hybrid systems, and are disclosed in U.S. Pat. Nos. 5,789,538; 5,925,523; 6,007,988; 6,013,453; 6,410,248; 6,140,466; 6,200,759; and 6,242,568, each of which is incorporated by reference.

A ZFN also includes a cleavage domain. The cleavage domain portion of the ZFNs may be obtained from any suitable endonuclease or exonuclease such as restriction endonucleases and homing endonucleases. See, for example, Belfort & Roberts, 1997, Homing endonucleases: keeping the house in order, Nucleic Acids Res 25(17):3379-3388. A cleavage domain may be derived from an enzyme that requires dimerization for cleavage activity. Two ZFNs may be required for cleavage, as each nuclease comprises a monomer of the active enzyme dimer. Alternatively, a single ZFN may comprise both monomers to create an active enzyme dimer. Restriction endonucleases present may be capable of sequence-specific binding and cleavage of DNA at or near the site of binding. Certain restriction enzymes (e.g., Type IIS) cleave DNA at sites removed from the recognition site and have separable binding and cleavage domains. For example, the Type IIS enzyme FokI, active as a dimer, catalyzes double-stranded cleavage of DNA, at 9 nucleotides from its recognition site on one strand and 13 nucleotides from its recognition site on the other. The FokI enzyme used in a ZFN may be considered a cleavage monomer. Thus, for targeted double-stranded cleavage using a FokI cleavage domain, two ZFNs, each comprising a FokI cleavage monomer, may be used to reconstitute an active enzyme dimer. See Wah, et al., 1998, Structure of FokI has implications for DNA cleavage, PNAS 95:10564-10569; U.S. Pat. Nos. 5,356,802; 5,436,150; 5,487,994; U.S. Pub. 2005/0064474; U.S. Pub. 2006/0188987; and U.S. Pub. 2008/0131962, each incorporated by reference.

Virus targeting using ZFN may include introducing at least one donor polynucleotide comprising a sequence into the cell. A donor polynucleotide preferably includes the sequence to be introduced flanked by an upstream and downstream sequence that share sequence similarity with either side of the site of integration in the chromosome. The upstream and downstream sequences in the donor polynucleotide are selected to promote recombination between the chromosomal sequence of interest and the donor polynucleotide. Typically, the donor polynucleotide will be DNA. The donor polynucleotide may be a DNA plasmid, a bacterial artificial chromosome (BAC), a yeast artificial chromosome (YAC), a viral vector, a linear piece of DNA, a PCR fragment, a naked nucleic acid, and may employ a delivery vehicle such as a liposome. The sequence of the donor polynucleotide may include exons, introns, regulatory sequences, or combinations thereof. The double stranded break is repaired via homologous recombination with the donor polynucleotide such that the desired sequence is integrated into the chromosome. In the ZFN-mediated process, a double stranded break introduced into the target sequence by the ZFN is repaired, via homologous recombination with the exchange polynucleotide, such that the sequence in the exchange polynucleotide may be exchanged with a portion of the target sequence. The presence of the double stranded break facilitates homologous recombination and repair of the break. The exchange polynucleotide may be physically integrated or, alternatively, the exchange polynucleotide may be used as a template for repair of the break, resulting in the exchange of the sequence information in the exchange polynucleotide with the sequence information in that portion of the target sequence. Thus, a portion of the viral nucleic acid may be converted to the sequence of the exchange polynucleotide. ZFN methods can include using a vector to deliver a nucleic acid molecule encoding a ZFN and, optionally, at least one exchange polynucleotide or at least one donor polynucleotide to the infected cell.

Meganucleases are endodeoxyribonucleases characterized by a large recognition site (double-stranded DNA sequences of 12 to 40 base pairs); as a result this site generally occurs only once in any given genome. For example, the 18-base pair sequence recognized by the I-SceI meganuclease would on average require a genome twenty times the size of the human genome to be found once by chance (although sequences with a single mismatch occur about three times per human-sized genome). Meganucleases are therefore considered to be the most specific naturally occurring restriction enzymes. Meganucleases can be divided into five families based on sequence and structure motifs: LAGLIDADG, GIY-YIG, HNH, His-Cys box and PD-(D/E)XK. The most well studied family is that of the LAGLIDADG proteins, which have been found in all kingdoms of life, generally encoded within introns or inteins although freestanding members also exist. The sequence motif, LAGLIDADG, represents an essential element for enzymatic activity. Some proteins contained only one such motif, while others contained two; in both cases the motifs were followed by ˜75-200 amino acid residues having little to no sequence similarity with other family members. Crystal structures illustrates mode of sequence specificity and cleavage mechanism for the LAGLIDADG family: (i) specificity contacts arise from the burial of extended β-strands into the major groove of the DNA, with the DNA binding saddle having a pitch and contour mimicking the helical twist of the DNA; (ii) full hydrogen bonding potential between the protein and DNA is never fully realized; (iii) cleavage to generate the characteristic 4-nt 3′-OH overhangs occurs across the minor groove, wherein the scissile phosphate bonds are brought closer to the protein catalytic core by a distortion of the DNA in the central “4-base” region; (iv) cleavage occurs via a proposed two-metal mechanism, sometimes involving a unique “metal sharing” paradigm; (v) and finally, additional affinity and/or specificity contacts can arise from “adapted” scaffolds, in regions outside the core a/0 fold. See Silva et al., 2011, Meganucleases and other tools for targeted genome engineering, Curr Gene Ther 11(1):11-27, incorporated by reference.

In some embodiments of the invention, a template sequence is inserted into the genome. In order to introduce nucleotide modifications to genomic DNA, a DNA repair template containing the desired sequence must be present during homology directed repair (HDR). The DNA template is normally transfected into the cell along with the gRNA/Cas9. The length and binding position of each homology arm is dependent on the size of the change being introduced. In the presence of a suitable template, HDR can introduce significant changes at the Cas9 induced double strand break.

Some embodiments of the invention may utilize modified version of a nuclease. Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. Similar to the inactive dCas9 (RuvC- and HNH-), a Cas9 nickase is still able to bind DNA based on gRNA specificity, though nickases will only cut one of the DNA strands. The majority of CRISPR plasmids are derived from S. pyogenes and the RuvC domain can be inactivated by a D10A mutation and the HNH domain can be inactivated by an H840A mutation.

A single-strand break, or nick, is normally quickly repaired through the HDR pathway, using the intact complementary DNA strand as the template. However, two proximal, opposite strand nicks introduced by a Cas9 nickase are treated as a double strand break, in what is often referred to as a ‘double nick’ or ‘dual nickase’ CRISPR system. A double-nick induced double strain break can be repaired by either NHEJ or HDR depending on the desired effect on the gene target. At these double strain breaks, insertions and deletions are caused by the CRISPR/Cas9 complex. In an aspect of the invention, a deletion is caused by positioning two double strand breaks proximate to one another, thereby causing a fragment of the genome to be deleted.

iii. Targeting Sequence

A nuclease may use the targeting specificity of a guide RNA (gRNA). As discussed below, guide RNAs or single guide RNAs are specifically designed to target a virus genome. As used herein targeting sequence can mean any combination of gRNA, crRNA, tracrRNA, sgRNA, and others. A CRISPR/Cas9 gene editing complex of the invention works optimally with a guide RNA that targets the viral genome. Guide RNA (gRNA) (which includes single guide RNA (sgRNA), crisprRNA (crRNA), transactivating RNA (tracrRNA), any other targeting oligo, or any combination thereof) leads the CRISPR/Cas9 complex to the viral genome in order to cause viral genomic disruption. In an aspect of the invention, CRISPR/Cas9/gRNA complexes are designed to target specific viruses within a cell. It should be appreciated that any virus can be targeted using the composition of the invention. Identification of specific regions of the virus genome aids in development and designing of CRISPR/Cas9/gRNA complexes.

In an aspect of the invention, the CRISPR/Cas9/gRNA complexes are designed to target latent viruses within a cell. Once transfected within a cell, the CRISPR/Cas9/gRNA complexes cause repeated insertions or deletions to render the genome incapacitated, or due to number of insertions or deletions, the probability of repair is significantly reduced.

As an example, we inactivated the Epstein-Barr virus (EBV), also called human herpesvirus 4 (HHV-4) in cells using a CRISPR/Cas9/gRNA complex. EBV is a virus of the herpes family, and is one of the most common viruses in humans. The virus is approximately 122 nm to 180 nm in diameter and is composed of a double helix of DNA wrapped in a protein capsid. In this example, the Raji cell line serves as an appropriate in vitro model. The Raji cell line is the first continuous human cell line from hematopoietic origin and cell lines produce an unusual strain of Epstein-Barr virus while being one of the most extensively studied EBV models. To target the EBV genomes in the Raji cells, a CRISPR/Cas9 complex with specificity for EBV is needed.

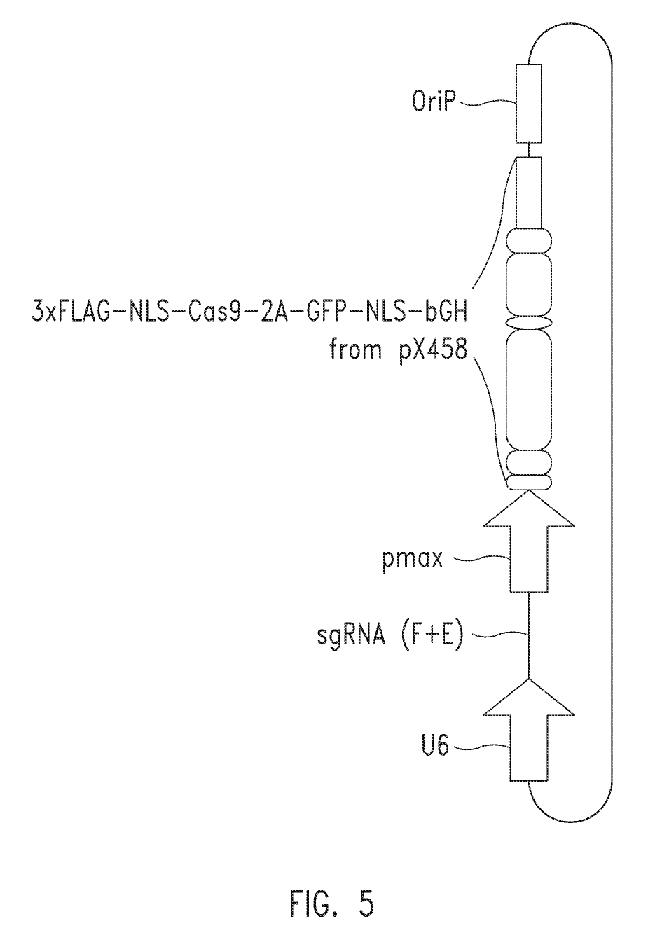

FIG. 5 shows a composition that includes an EGFP marker fused after the Cas9 protein.

The design of EBV-targeting CRISPR/Cas9 plasmids consisting of a U6 promoter driven chimeric guide RNA (sgRNA) and a ubiquitous promoter driven Cas9 that were obtained from Addgene, Inc. Commercially available guide RNAs and Cas9 nucleases may be used with the present invention. The EGFP marker fused after the Cas9 protein allowed selection of Cas9-positive cells.

Preferably guide RNAs are designed, whether or not commercially purchased, to target a specific part of an HPV genome. The target area in HPV is identified and guide RNA to target selected portions of the HPV genome are developed and incorporated into the composition of the invention. In an aspect of the invention, a reference genome of a particular strain of the virus is selected for guide RNA design.

In relation to EBV, for example, the reference genome from strain B95-8 was used as a design guide. Within a genome of interest, such as EBV, selected regions, or genes are targeted. For example, six regions can be targeted with seven guide RNA designs for different genome editing purposes.

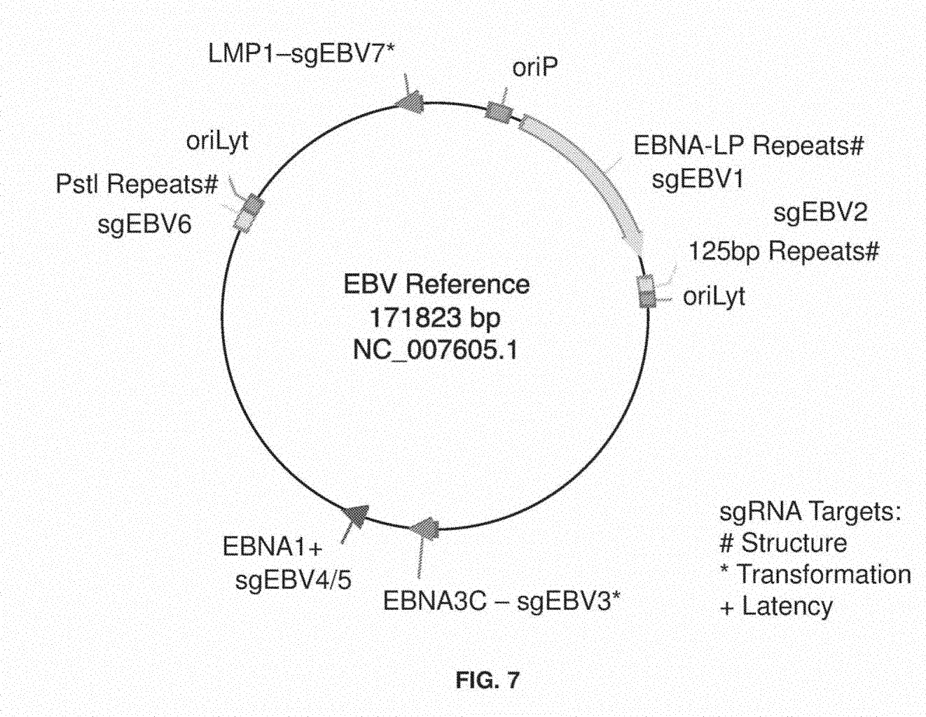

FIG. 7 is a diagram of an EBV genome, with structure-, transformation-, and latency-related targets called out. FIG. 7 additionally shows where sgEBV1, sgEBV2, sgEBV3, sgEBV4/5, sgEBV6, and sgEBV7 target the EBV genome.

FIG. 7 shows gRNA targets along a reference genome where # denotes structural targets, * denotes transformation-related targets, and + denotes latency-related targets.

In relation to EBV, EBNA1 is the only nuclear Epstein-Barr virus (EBV) protein expressed in both latent and lytic modes of infection. While EBNA1 is known to play several important roles in latent infection, EBNA1 is crucial for many EBV functions including gene regulation and latent genome replication. Therefore, guide RNAs sgEBV4 and sgEBV5 were selected to target both ends of the EBNA1 coding region in order to excise this whole region of the genome. These “structural” targets enable systematic digestion of the EBV genome into smaller pieces. EBNA3C and LMP1 are essential for host cell transformation, and guide RNAs sgEBV3 and sgEBV7 were designed to target the 5′ exons of these two proteins respectively.

iv. Introduce to Cell

Methods of the invention include introducing into an HPV-infected keratinocyte a nuclease and a sequence-specific targeting moiety. The nuclease is targeted to HPV nucleic acid by means of the sequence-specific targeting moiety where it then cleaves the viral nucleic acid without interfering with a host genome. Any suitable method can be used to deliver the nuclease to the infected cell or tissue. For example, the nuclease or the gene encoding the nuclease may be delivered by injection, orally, or by hydrodynamic delivery. The nuclease or the gene encoding the nuclease may be delivered to systematic circulation or may be delivered or otherwise localized to a specific tissue type. The nuclease or gene encoding the nuclease may be modified or programmed to be active under only certain conditions such as by using a tissue-specific promoter so that the encoded nuclease is preferentially or only transcribed in certain tissue types.

In some embodiments, specific CRISPR/Cas9/gRNA complexes are introduced into a cell. A guide RNA is designed to target at least one category of sequences of the viral genome. In addition to latent infections this invention can also be used to control actively replicating viruses by targeting the viral genome before it is packaged or after it is ejected.

In some embodiments, a cocktail of guide RNAs may be introduced into a cell. The guide RNAs are designed to target numerous categories of sequences of the viral genome. By targeting several areas along the genome, the double strand break at multiple locations fragments the genome, lowering the possibility of repair. Even with repair mechanisms, the large deletions render the virus incapacitated.

In some embodiments, several guide RNAs are added to create a cocktail to target different categories of sequences. For example, two, five, seven or eleven guide RNAs may be present in a CRISPR cocktail targeting three different categories of sequences. However, any number of gRNAs may be introduced into a cocktail to target categories of sequences. In preferred embodiments, the categories of sequences are important for genome structure, host cell transformation, and infection latency, respectively.

In some aspects of the invention, in vitro experiments allow for the determination of the most essential targets within a viral genome. For example, to understand the most essential targets for effective incapacitation of a genome, subsets of guide RNAs are transfected into model cells. Assays can determine which guide RNAs or which cocktail is the most effective at targeting essential categories of sequences.

For example, in the case of the EBV genome targeting, seven guide RNAs in the CRISPR cocktail targeted three different categories of sequences which are identified as being important for EBV genome structure, host cell transformation, and infection latency, respectively. To understand the most essential targets for effective EBV treatment, Raji cells were transfected with subsets of guide RNAs. Although sgEBV4/5 reduced the EBV genome by 85%, they could not suppress cell proliferation as effectively as the full cocktail (FIG. 14). Guide RNAs targeting the structural sequences (sgEBV1/2/6) could stop cell proliferation completely, despite not eliminating the full EBV load (26% decrease). Given the high efficiency of genome editing and the proliferation arrest, it was suspect that the residual EBV genome signature in sgEBV1/2/6 was not due to intact genomes but to free-floating DNA that has been digested out of the EBV genome, i.e. as a false positive.

Once CRISPR/Cas9/gRNA complexes are constructed, the complexes are introduced into a cell. It should be appreciated that complexes can be introduced into cells in an in vitro model or an in vivo model. In an aspect of the invention, CRISPR/Cas9/gRNA complexes are designed to not leave intact genomes of a virus after transfection and complexes are designed for efficient transfection.

Aspects of the invention allow for CRISPR/Cas9/gRNA to be transfected into cells by various methods, including viral vectors and non-viral vectors. Viral vectors may include retroviruses, lentiviruses, adenoviruses, and adeno-associated viruses. It should be appreciated that any viral vector may be incorporated into the present invention to effectuate delivery of the CRISPR/Cas9/gRNA complex into a cell. Some viral vectors may be more effective than others, depending on the CRISPR/Cas9/gRNA complex designed for digestion or incapacitation. In an aspect of the invention, the vectors contain essential components such as origin of replication, which is necessary for the replication and maintenance of the vector in the host cell.

In an aspect of the invention, viral vectors are used as delivery vectors to deliver the complexes into a cell. Use of viral vectors as delivery vectors are known in the art. See for example U.S. Pub. 2009/0017543 to Wilkes et al., the contents of which are incorporated by reference.

A retrovirus is a single-stranded RNA virus that stores its nucleic acid in the form of an mRNA genome (including the 5′ cap and 3′ PolyA tail) and targets a host cell as an obligate parasite. In some methods in the art, retroviruses have been used to introduce nucleic acids into a cell. Once inside the host cell cytoplasm the virus uses its own reverse transcriptase enzyme to produce DNA from its RNA genome, the reverse of the usual pattern, thus retro (backwards). This new DNA is then incorporated into the host cell genome by an integrase enzyme, at which point the retroviral DNA is referred to as a provirus. For example, the recombinant retroviruses such as the Moloney murine leukemia virus have the ability to integrate into the host genome in a stable fashion. They contain a reverse transcriptase that allows integration into the host genome. Retroviral vectors can either be replication-competent or replication-defective. In some embodiments of the invention, retroviruses are incorporated to effectuate transfection into a cell, however the CRISPR/Cas9/gRNA complexes are designed to target the viral genome.

In some embodiments of the invention, lentiviruses, which are a subclass of retroviruses, are used as viral vectors. Lentiviruses can be adapted as delivery vehicles (vectors) given their ability to integrate into the genome of non-dividing cells, which is the unique feature of lentiviruses as other retroviruses can infect only dividing cells. The viral genome in the form of RNA is reverse-transcribed when the virus enters the cell to produce DNA, which is then inserted into the genome at a random position by the viral integrase enzyme. The vector, now called a provirus, remains in the genome and is passed on to the progeny of the cell when it divides.

As opposed to lentiviruses, adenoviral DNA does not integrate into the genome and is not replicated during cell division. Adenovirus and the related AAV may be used as delivery vectors since they do not integrate into the host's genome. In some aspects of the invention, only the viral genome to be targeted is effected by the CRISPR/Cas9/gRNA complexes, and not the host's cells. Adeno-associated virus (AAV) is a small virus that infects humans and some other primate species. AAV can infect both dividing and non-dividing cells and may incorporate its genome into that of the host cell. For example, because of its potential use as a gene therapy vector, researchers have created an altered AAV called self-complementary adeno-associated virus (scAAV). Whereas AAV packages a single strand of DNA and requires the process of second-strand synthesis, scAAV packages both strands which anneal together to form double stranded DNA. By skipping second strand synthesis scAAV allows for rapid expression in the cell. Otherwise, scAAV carries many characteristics of its AAV counterpart. Addtionally or alternatively, methods and compositions of the invention may use herpesvirus, poxvirus, alphavirus, or vaccinia virus as a means of delivery vectors.

In certain embodiments of the invention, non-viral vectors may be used to effectuate transfection. Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam and Lipofectin). Cationic and neutral lipids that are suitable for efficient receptor-recognition lipofection of polynucleotides include those described in U.S. Pat. No. 7,166,298 to Jessee or U.S. Pat. No. 6,890,554 to Jesse, the contents of each of which are incorporated by reference. Delivery can be to cells (e.g. in vitro or ex vivo administration) or target tissues (e.g. in vivo administration).

Synthetic vectors are typically based on cationic lipids or polymers which can complex with negatively charged nucleic acids to form particles with a diameter in the order of 100 nm. The complex protects nucleic acid from degradation by nuclease. Moreover, cellular and local delivery strategies have to deal with the need for internalization, release, and distribution in the proper subcellular compartment. Systemic delivery strategies encounter additional hurdles, for example, strong interaction of cationic delivery vehicles with blood components, uptake by the reticuloendothelial system, kidney filtration, toxicity and targeting ability of the carriers to the cells of interest. Modifying the surfaces of the cationic non-virals can minimize their interaction with blood components, reduce reticuloendothelial system uptake, decrease their toxicity and increase their binding affinity with the target cells. Binding of plasma proteins (also termed opsonization) is the primary mechanism for RES to recognize the circulating nanoparticles. For example, macrophages, such as the Kupffer cells in the liver, recognize the opsonized nanoparticles via the scavenger receptor.

In some embodiments of the invention, non-viral vectors are modified to effectuate targeted delivery and transfection. PEGylation (i.e. modifying the surface with polyethyleneglycol) is the predominant method used to reduce the opsonization and aggregation of non-viral vectors and minimize the clearance by reticuloendothelial system, leading to a prolonged circulation lifetime after intravenous (i.v.) administration. PEGylated nanoparticles are therefore often referred as “stealth” nanoparticles. The nanoparticles that are not rapidly cleared from the circulation will have a chance to encounter infected cells.

However, PEG on the surface can decrease the uptake by target cells and reduce the biological activity. Therefore, to attach targeting ligand to the distal end of the PEGylated component is necessary; the ligand is projected beyond the PEG “shield” to allow binding to receptors on the target cell surface. When cationic liposome is used as gene carrier, the application of neutral helper lipid is helpful for the release of nucleic acid, besides promoting hexagonal phase formation to enable endosomal escape. In some embodiments of the invention, neutral or anionic liposomes are developed for systemic delivery of nucleic acids and obtaining therapeutic effect in experimental animal model. Designing and synthesizing novel cationic lipids and polymers, and covalently or noncovalently binding gene with peptides, targeting ligands, polymers, or environmentally sensitive moieties also attract many attentions for resolving the problems encountered by non-viral vectors. The application of inorganic nanoparticles (for example, metallic nanoparticles, iron oxide, calcium phosphate, magnesium phosphate, manganese phosphate, double hydroxides, carbon nanotubes, and quantum dots) in delivery vectors can be prepared and surface-functionalized in many different ways.

In some embodiments, the complexes are conjugated to nano-systems for systemic therapy, such as liposomes, albumin-based particles, PEGylated proteins, biodegradable polymer-drug composites, polymeric micelles, dendrimers, among others. See Davis et al., 2008, Nanotherapeutic particles: an emerging treatment modality for cancer, Nat Rev Drug Discov. 7(9):771-782, incorporated by reference. Long circulating macromolecular carriers such as liposomes, can exploit the enhanced permeability and retention effect for preferential extravasation from tumor vessels. In certain embodiments, the complexes of the invention are conjugated to or encapsulated into a liposome or polymerosome for delivery to a cell. For example, liposomal anthracyclines have achieved highly efficient encapsulation, and include versions with greatly prolonged circulation such as liposomal daunorubicin and pegylated liposomal doxorubicin. See Krishna et al., Carboxymethylcellulose-sodium based transdermal drug delivery system for propranolol, J Pharm Pharmacol. 1996 April; 48(4):367-70.

Liposomal delivery systems provide stable formulation, provide improved pharmacokinetics, and a degree of ‘passive’ or ‘physiological’ targeting to tissues. Encapsulation of hydrophilic and hydrophobic materials, such as potential chemotherapy agents, are known. See for example U.S. Pat. No. 5,466,468 to Schneider, which discloses parenterally administrable liposome formulation comprising synthetic lipids; U.S. Pat. No. 5,580,571, to Hostetler et al. which discloses nucleoside analogues conjugated to phospholipids; U.S. Pat. No. 5,626,869 to Nyqvist, which discloses pharmaceutical compositions wherein the pharmaceutically active compound is heparin or a fragment thereof contained in a defined lipid system comprising at least one amphiphatic and polar lipid component and at least one nonpolar lipid component.

Liposomes and polymerosomes can contain a plurality of solutions and compounds. In certain embodiments, the complexes of the invention are coupled to or encapsulated in polymersomes. As a class of artificial vesicles, polymersomes are tiny hollow spheres that enclose a solution, made using amphiphilic synthetic block copolymers to form the vesicle membrane. Common polymersomes contain an aqueous solution in their core and are useful for encapsulating and protecting sensitive molecules, such as drugs, enzymes, other proteins and peptides, and DNA and RNA fragments. The polymersome membrane provides a physical barrier that isolates the encapsulated material from external materials, such as those found in biological systems. Polymerosomes can be generated from double emulsions by known techniques, see Lorenceau et al., 2005, Generation of Polymerosomes from Double-Emulsions, Langmuir 21(20):9183-6, incorporated by reference.

Aspects of the invention provide for numerous uses of delivery vectors. Selection of the delivery vector is based upon the cell or tissue targeted and the specific makeup of the CRISPR/Cas9/gRNA. For example, in the EBV example discussed above, since lymphocytes are known for being resistant to lipofection, nucleofection (a combination of electrical parameters generated by a device called Nucleofector, with cell-type specific reagents to transfer a substrate directly into the cell nucleus and the cytoplasm) was necessitated for DNA delivery into the Raji cells. The Lonza pmax promoter drives Cas9 expression as it offered strong expression within Raji cells. 24 hours after nucleofection, obvious EGFP signals were observed from a small proportion of cells through fluorescent microscopy. The EGFP-positive cell population decreased dramatically, however, <10% transfection efficiency 48 hours after nucleofection was measured.

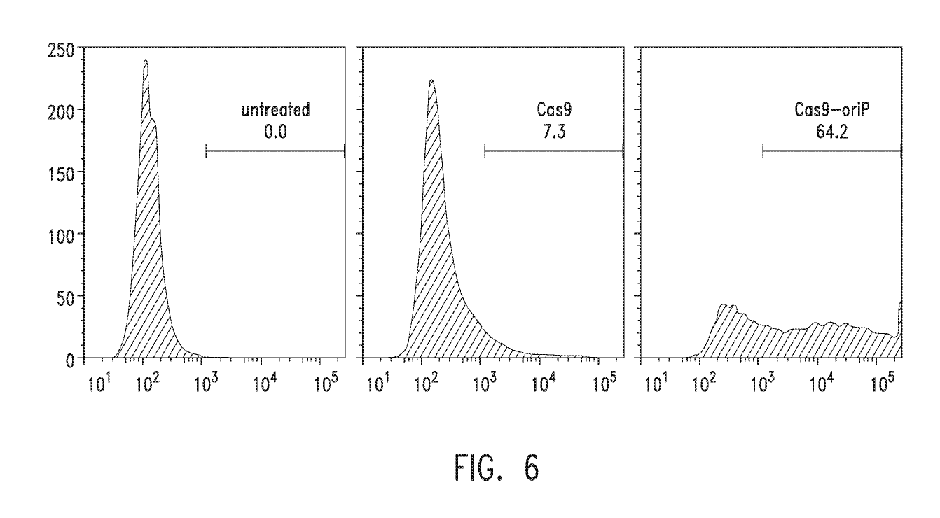

FIG. 6 shows the effect of oriP on transfection efficiency in Raji cells. A CRISPR plasmid that included the EBV origin of replication sequence, oriP yielded a transfection efficiency >60%.

Aspects of the invention use the CRISPR/Cas9/gRNA complexes and targeted delivery. Common known pathways include transdermal, transmucal, nasal, ocular and pulmonary routes. Drug delivery systems may include liposomes, proliposomes, microspheres, gels, prodrugs, cyclodextrins, etc. Aspects of the invention utilize nanoparticles composed of biodegradable polymers to be transferred into an aerosol for targeting of specific sites or cell populations in the lung, providing for the release of the drug in a predetermined manner and degradation within an acceptable period of time. Controlled-release technology (CRT), such as transdermal and transmucosal controlled-release delivery systems, nasal and buccal aerosol sprays, drug-impregnated lozenges, encapsulated cells, oral soft gels, iontophoretic devices to administer drugs through skin, and a variety of programmable, implanted drug-delivery devices are used in conjunction with the complexes of the invention of accomplishing targeted and controlled delivery.

v. Cut Viral Nucleic Acid

Once inside the cell, the CRISPR/Cas9/gRNA complexes target the viral genome. In an aspect of the invention, the complexes are targeted to viral genomes. In addition to latent infections this invention can also be used to control actively replicating viruses by targeting the viral genome before it is packaged or after it is ejected. In some embodiments, methods and compositions of the invention use a nuclease such as Cas9 to target latent viral genomes, thereby reducing the chances of proliferation.

FIG. 3 shows the results of successfully cleaving the HPV genome using Cas9 endonuclease, a gRNA for E6, and a gRNA for E7. The nuclease forms a complex with the gRNA (e.g., crRNA+tracrRNA or sgRNA). The complex cuts the viral nucleic acid in a targeted fashion to incapacitate the viral genome. The Cas9 endonuclease causes a double strand break in the viral genome. By targeted several locations along the viral genome and causing not a single strand break, but a double strand break, the genome is effectively cut a several locations along the genome. In a preferred embodiment, the double strand breaks are designed so that small deletions are caused, or small fragments are removed from the genome so that even if natural repair mechanisms join the genome together, the genome is render incapacitated.

After introduction into a keratinocyte, the CRISPR/Cas9/gRNA complexes act on the HPV genome, genes, transcripts, or other viral nucleic acid. The double-strand DNA breaks generated by CRISPR are repaired with small deletions. These deletions will disrupt the protein coding and hence create knockout effects.

The nuclease, or a gene encoding the nuclease, may be delivered into an infected keratinocyte by transfection. For example, the infected cell can be transfected with DNA that encodes Cas9 and gRNA (on a single piece or separate pieces). The gRNAs are designed to localize the Cas9 endonuclease at one or several locations along the viral genome. The Cas9 endonuclease causes double strand breaks in the genome, causing small fragments to be deleted from the viral genome. Even with repair mechanisms, the deletions render the viral genome incapacitated.

vi. Host Genome

It will be appreciated that method and compositions of the invention can be used to target viral nucleic acid without interfering with host genetic material. Methods and compositions of the invention employ a targeting moiety such as a guide RNA that has a sequence that hybridizes to a target within the viral sequence. Methods and compositions of the invention may further use a targeted nuclease such as the cas9 enzyme, or a vector encoding such a nuclease, which uses the gRNA to bind exclusively to the viral genome and make double stranded cuts, thereby removing the viral sequence from the host.

Where the targeting moiety includes a guide RNA, the sequence for the gRNA, or the guide sequence, can be determined by examination of the viral sequence to find regions of about 20 nucleotides that are adjacent to a protospacer adjacent motif (PAM) and that do not also appear in the host genome adjacent to the protospacer motif.

Preferably a guide sequence that satisfies certain similarity criteria (e.g., at least 60% identical with identity biased toward regions closer to the PAM) so that a gRNA/cas9 complex made according to the guide sequence will bind to and digest specified features or targets in the viral sequence without interfering with the host genome. Preferably, the guide RNA corresponds to a nucleotide string next to a protospacer adjacent motif (PAM) (e.g., NGG, where N is any nucleotide) in the viral sequence. Preferably, the host genome lacks any region that (1) matches the nucleotide string according to a predetermined similarity criteria and (2) is also adjacent to the PAM. The predetermined similarity criteria may include, for example, a requirement of at least 12 matching nucleotides within 20 nucleotides 5′ to the PAM and may also include a requirement of at least 7 matching nucleotides within 10 nucleotides 5′ to the PAM. An annotated viral genome (e.g., from GenBank) may be used to identify features of the viral sequence and finding the nucleotide string next to a protospacer adjacent motif (PAM) in the viral sequence within a selected feature (e.g., a viral replication origin, a terminal repeat, a replication factor binding site, a promoter, a coding sequence, or a repetitive region) of the viral sequence. The viral sequence and the annotations may be obtained from a genome database.

Where multiple candidate gRNA targets are found in the viral genome, selection of the sequence to be the template for the guide RNA may favor the candidate target closest to, or at the 5′ most end of, a targeted feature as the guide sequence. The selection may preferentially favor sequences with neutral (e.g., 40% to 60%) GC content. Additional background regarding the RNA-directed targeting by endonuclease is discussed in U.S. Pub. 2015/0050699; U.S. Pub. 20140356958; U.S. Pub. 2014/0349400; U.S. Pub. 2014/0342457; U.S. Pub. 2014/0295556; and U.S. Pub. 2014/0273037, the contents of each of which are incorporated by reference for all purposes. Due to the existence of human genomes background in the infected cells, a set of steps are provided to ensure high efficiency against the viral genome and low off-target effect on the human genome. Those steps may include (1) target selection within viral genome, (2) avoiding PAM+target sequence in host genome, (3) methodologically selecting viral target that is conserved across strains, (4) selecting target with appropriate GC content, (5) control of nuclease expression in cells, (6) vector design, (7) validation assay, others and various combinations thereof. A targeting moiety (such as a guide RNA) preferably binds to targets within certain categories such as (i) latency related targets, (ii) infection and symptom related targets, and (iii) structure related targets.

A first category of targets for gRNA includes latency-related targets. The viral genome requires certain features in order to maintain the latency. These features include, but not limited to, master transcription regulators, latency-specific promoters, signaling proteins communicating with the host cells, etc. If the host cells are dividing during latency, the viral genome requires a replication system to maintain genome copy level. Viral replication origin, terminal repeats, and replication factors binding to the replication origin are great targets. Once the functions of these features are disrupted, the viruses may reactivate, which can be treated by conventional antiviral therapies.

A second category of targets for gRNA includes infection-related and symptom-related targets. Virus produces various molecules to facilitate infection. Once gained entrance to the host cells, the virus may start lytic cycle, which can cause cell death and tissue damage (HBV). In certain cases, such as HPV16, cell products (E6 and E7 proteins) can transform the host cells and cause cancers. Disrupting the key genome sequences (promoters, coding sequences, etc) producing these molecules can prevent further infection, and/or relieve symptoms, if not curing the disease.

A third category of targets for gRNA includes structure-related targets. Viral genome may contain repetitive regions to support genome integration, replication, or other functions. Targeting repetitive regions can break the viral genome into multiple pieces, which physically destroys the genome.

Where the nuclease is a cas protein, the targeting moiety is a guide RNA. Each cas protein requires a specific PAM next to the targeted sequence (not in the guide RNA). This is the same as for human genome editing. The current understanding the guide RNA/nuclease complex binds to PAM first, then searches for homology between guide RNA and target genome. Sternberg et al., 2014, DNA interrogation by the CRISPR RNA-guided endonuclease Cas9, Nature 507(7490):62-67. Once recognized, the DNA is digested 3-nt upstream of PAM. These results suggest that off-target digestion requires PAM in the host DNA, as well as high affinity between guide RNA and host genome right before PAM.

It may be preferable to use a targeting moiety that targets portions of the viral genome that are highly conserved. Viral genomes are much more variable than human genomes. In order to target different strains, the guide RNA will preferably target conserved regions. As PAM is important to initial sequence recognition, it is also essential to have PAM in the conserved region.

In a preferred embodiment, methods of the invention are used to deliver a nucleic acid to cells. The nucleic acid delivered to the cells may include a gRNA having the determined guide sequence or the nucleic acid may include a vector, such as a plasmid, that encodes an enzyme that will act against the target genetic material. Expression of that enzyme allows it to degrade or otherwise interfere with the target genetic material. The enzyme may be a nuclease such as the Cas9 endonuclease and the nucleic acid may also encode one or more gRNA having the determined guide sequence.

The gRNA targets the nuclease to the target genetic material. Where the target genetic material includes the genome of a virus, gRNAs complementary to parts of that genome can guide the degredation of that genome by the nuclease, thereby preventing any further replication or even removing any intact viral genome from the cells entirely. By these means, latent viral infections can be targeted for eradication.

The host cells may grow at different rate, based on the specific cell type. High nuclease expression is necessary for fast replicating cells, whereas low expression help avoiding off-target cutting in non-infected cells. Control of nuclease expression can be achieved through several aspects. If the nuclease is expressed from a vector, having the viral replication origin in the vector can increase the vector copy number dramatically, only in the infected cells. Each promoter has different activities in different tissues. Gene transcription can be tuned by choosing different promoters. Transcript and protein stability can also be tuned by incorporating stabilizing or destabilizing (ubiquitin targeting sequence, etc) motif into the sequence.

Specific promoters may be used for the gRNA sequence, the nuclease (e.g., cas9), other elements, or combinations thereof. For example, in some embodiments, the gRNA is driven by a U6 promoter. A vector may be designed that includes a promoter for protein expression (e.g., using a promoter as described in the vector sold under the trademark PMAXCLONING by Lonza Group Ltd (Basel, Switzerland). A vector may be a plasmid (e.g., created by synthesis instrument 255 and recombinant DNA lab equipment). In certain embodiments, the plasmid includes a U6 promoter driven gRNA or chimeric guide RNA (sgRNA) and a ubiquitous promoter-driven cas9. Optionally, the vector may include a marker such as EGFP fused after the cas9 protein to allow for later selection of cas9+ cells. It is recognized that cas9 can use a gRNA (similar to the CRISPR RNA (crRNA) of the original bacterial system) with a complementary trans-activating crRNA (tracrRNA) to target viral sequences complementary to the gRNA. It has also been shown that cas9 can be programmed with a single RNA molecule, a chimera of the gRNA and tracrRNA. The single guide RNA (sgRNA) can be encoded in a plasmid and transcription of the sgRNA can provide the programming of cas9 and the function of the tracrRNA. See Jinek, 2012, A programmable dual-RNA-guided DNA endonuclease in adaptive bacterial immunity, Science 337:816-821 and especially FIG. 5A therein for background.

Using the above principles, methods and compositions of the invention may be used to target viral nucleic acid in an infected host without adversely influencing the host genome.

For additional background see Hsu, 2013, DNA targeting specificity of RNA-guided Cas9 nucleases, Nature Biotechnology 31(9):827-832; and Jinek, 2012, A programmable dual-RNA-guided DNA endonuclease in adaptive bacterial immunity, Science 337:816-821, the contents of each of which are incorporated by reference. Since the targeted locations are selected to be within certain categories such as (i) latency related targets, (ii) infection and symptom related targets, or (iii) structure related targets, cleavage of those sequences inactivates the virus and removes it from the host. Since the targeting RNA (the gRNA or sgRNA) is designed to satisfy according to similarity criteria that matches the target in the viral genetic sequence without any off-target matching the host genome, the latent viral genetic material is removed from the host without any interference with the host genome.

vii. Composition

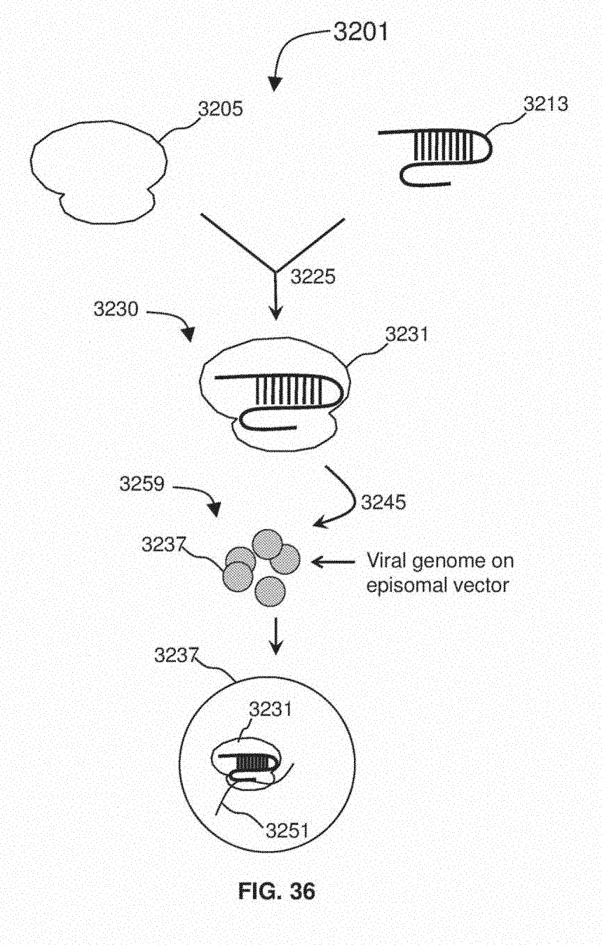

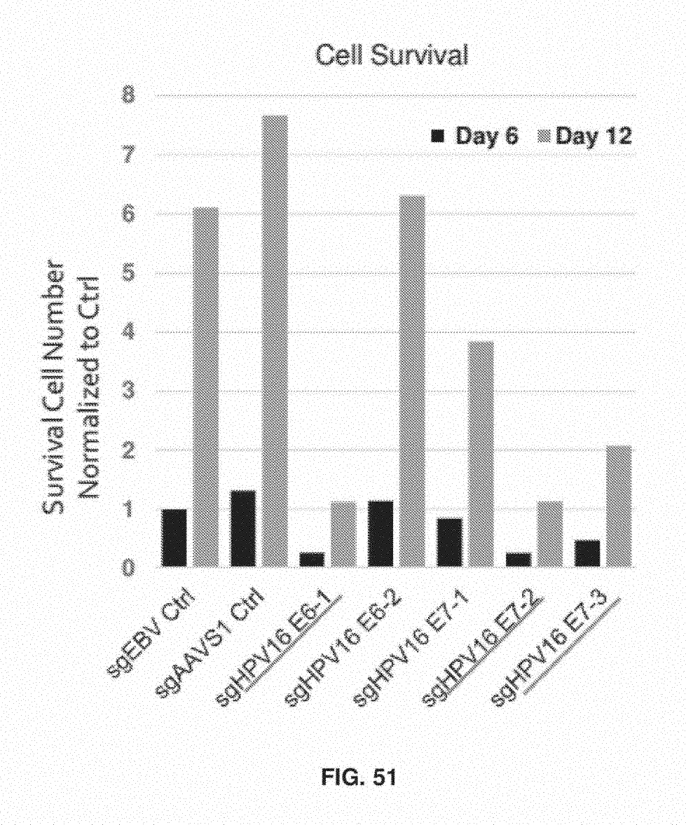

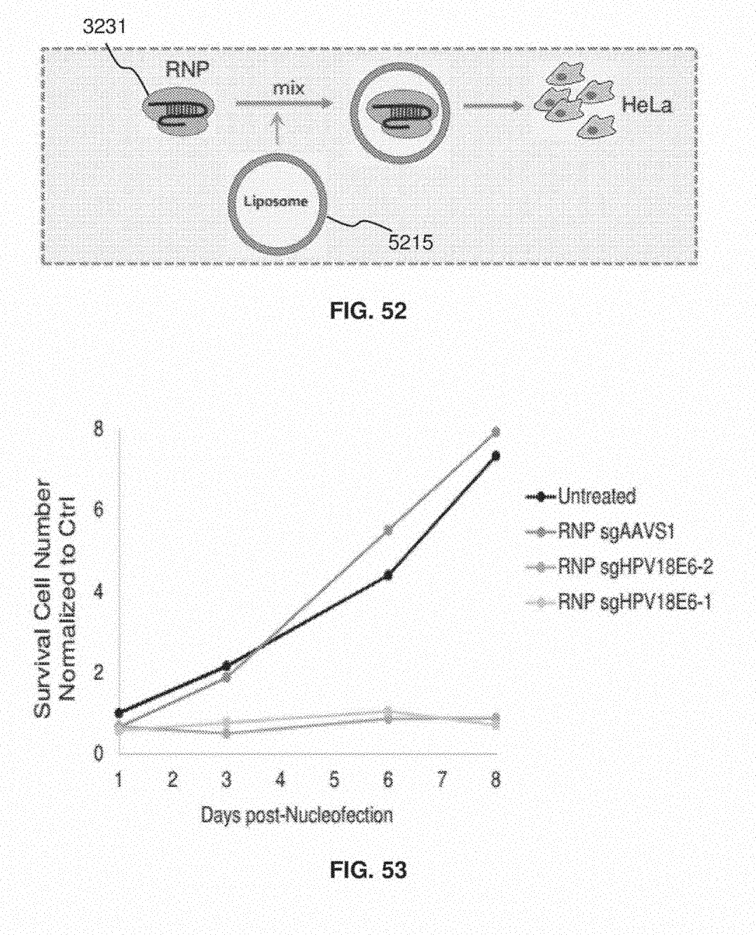

FIG. 36 depicts a composition 3230 that includes a ribonucleoprotein (RNP) 3231 comprising an RNA-guided nuclease 3205 and an RNA 3213 with a portion complementary to a target site within a viral nucleic acid of the virus. The RNP is preferably extra-cellular in that it exists in active form in solution outside of any cell. The RNA-guided nuclease 3205 may be a CRISPR-associated protein such as Cas9 or Cpf1. FIG. 52 shows a liposome 5215 enveloping the RNP 3231. FIG. 53 shows that the RNP 3231 enveloped in the liposome 5215 provides an embodiment of the composition that when delivered to cells infected with human papillomavirus (HPV), cleaves viral nucleic acid of the HPV within early genes, specifically E6 and E7. As shown by FIG. 53 and FIG. 51, when multiple RNPs are delivered with guide RNAs targeting multiple sites within the early genes (E6 and E7), the composition is effective in killing HPV+ cancer cells. FIG. 36 diagrams a method 3201 of cleaving foreign nucleic acid within cells (effectively removing that foreign nucleic acid as the cleavage products likely enter metabolic pathways). The method includes delivering to cells 3259 or tissue in vitro or in a patient a composition 3230 that includes an extra-cellular RNP 3231 according to any of the embodiments described above and cleaving viral nucleic acid with the RNP.

In some embodiments, the invention provides a composition 3230 for topical application (e.g., in vivo, directly to skin of a person). The composition may be applied superficially (e.g., topically). The composition provides a nuclease 3205 or gene therefore and includes a pharmaceutically acceptable diluent, adjuvant, or carrier. Preferably, a carrier used in accordance with the subject invention is approved for animal or human use by a competent governmental agency, such as the US Food and Drug Administration (FDA) or the like. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. These formulations contain from about 0.01% to about 100%, preferably from about 0.01% to about 90% of the MFB extract, the balance (from about 0% to about 99.99%, preferably from about 10% to about 99.99% of an acceptable carrier or other excipients. A more preferred formulation contains up to about 10% MFB extract and about 90% or more of the carrier or excipient, whereas a typical and most preferred composition contains about 5% MFB extract and about 95% of the carrier or other excipients. Formulations are described in a number of sources that are well known and readily available to those skilled in the art.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

### EXAMPLES

### Example 1: Targeting EBV

Burkitt's lymphoma cell lines Raji, Namalwa, and DG-75 were obtained from ATCC and cultured in RPMI 1640 supplemented with 10% FBS and PSA, following ATCC recommendation. Human primary lung fibroblast IMR-90 was obtained from Coriell and cultured in Advanced DMEM/F-12 supplemented with 10% FBS and PSA.

Plasmids consisting of a U6 promoter driven chimeric guide RNA (sgRNA) and a ubiquitous promoter driven Cas9 were obtained from addgene, as described by Cong L et al. (2013) Multiplex Genome Engineering Using CRISPR/Cas Systems. Science 339:819-823. An EGFP marker fused after the Cas9 protein allowed selection of Cas9-positive cells (FIG. 5). We adapted a modified chimeric guide RNA design for more efficient Pol-III transcription and more stable stem-loop structure (Chen B et al. (2013) Dynamic Imaging of Genomic Loci in Living Human Cells by an Optimized CRISPR/Cas System. Cell 155:1479-1491).

We obtained pX458 from Addgene, Inc. A modified CMV promoter with a synthetic intron (pmax) was PCR amplified from Lonza control plasmid pmax-GFP. A modified guide RNA sgRNA(F+E) was ordered from IDT. EBV replication origin oriP was PCR amplified from B95-8 transformed lymphoblastoid cell line GM12891. We used standard cloning protocols to clone pmax, sgRNA(F+E) and oriP to pX458, to replace the original CAG promoter, sgRNA and f1 origin. We designed EBV sgRNA based on the B95-8 reference, and ordered DNA oligos from IDT. The original sgRNA place holder in pX458 serves as the negative control.

Lymphocytes are known for being resistant to lipofection, and therefore we used nucleofection for DNA delivery into Raji cells. We chose the Lonza pmax promoter to drive Cas9 expression as it offered strong expression within Raji cells. We used the Lonza Nucleofector II for DNA delivery. 5 million Raji or DG-75 cells were transfected with 5 ug plasmids in each 100-ul reaction. Cell line Kit V and program M-013 were used following Lonza recommendation. For IMR-90, 1 million cells were transfected with 5 ug plasmids in 100 ul Solution V, with program T-030 or X-005. 24 hours after nucleofection, we observed obvious EGFP signals from a small proportion of cells through fluorescent microscopy. The EGFP-positive cell population decreased dramatically after that, however, and we measured <10% transfection efficiency 48 hours after nucleofection (FIG. 6). We attributed this transfection efficiency decrease to the plasmid dilution with cell division. To actively maintain the plasmid level within the host cells, we redesigned the CRISPR plasmid to include the EBV origin of replication sequence, oriP. With active plasmid replication inside the cells, the transfection efficiency rose to >60% (FIG. 6).

To design guide RNA targeting the EBV genome, we relied on the EBV reference genome from strain B95-8. We targeted six regions with seven guide RNA designs for different genome editing purposes. The guide RNAs are listed in Table 51 in Wang and Quake, 2014, RNA-guided endonuclease provides a therapeutic strategy to cure latent herpesviridae infection, PNAS 111(36):13157-13162 and in the Supporting Information to that article published online at the PNAS website, and the contents of both of those documents are incorporated by reference for all purposes.

EBNA1 is crucial for many EBV functions including gene regulation and latent genome replication. We targeted guide RNA sgEBV4 and sgEBV5 to both ends of the EBNA1 coding region in order to excise this whole region of the genome. Guide RNAs sgEBV1, 2 and 6 fall in repeat regions, so that the success rate of at least one CRISPR cut is multiplied. These “structural” targets enable systematic digestion of the EBV genome into smaller pieces. EBNA3C and LMP1 are essential for host cell transformation, and we designed guide RNAs sgEBV3 and sgEBV7 to target the 5′ exons of these two proteins respectively.

EBV Genome Editing.

The double-strand DNA breaks generated by CRISPR are repaired with small deletions. These deletions will disrupt the protein coding and hence create knockout effects. SURVEYOR assays confirmed efficient editing of individual sites.

FIG. 32 represents SURVEYOR assay of EBV CRISPR (lanes numbered from left to right: Lane 1: NEB 100 bp ladder; Lane 2: sgEBV1 control; Lane 3: sgEBV1; Lane 4: sgEBV5 control; Lane 5: sgEBV5; Lane 6: sgEBV7 control; Lane 7: sgEBV7; Lane 8: sgEBV4).

Beyond the independent small deletions induced by each guide RNA, large deletions between targeting sites can systematically destroy the EBV genome.

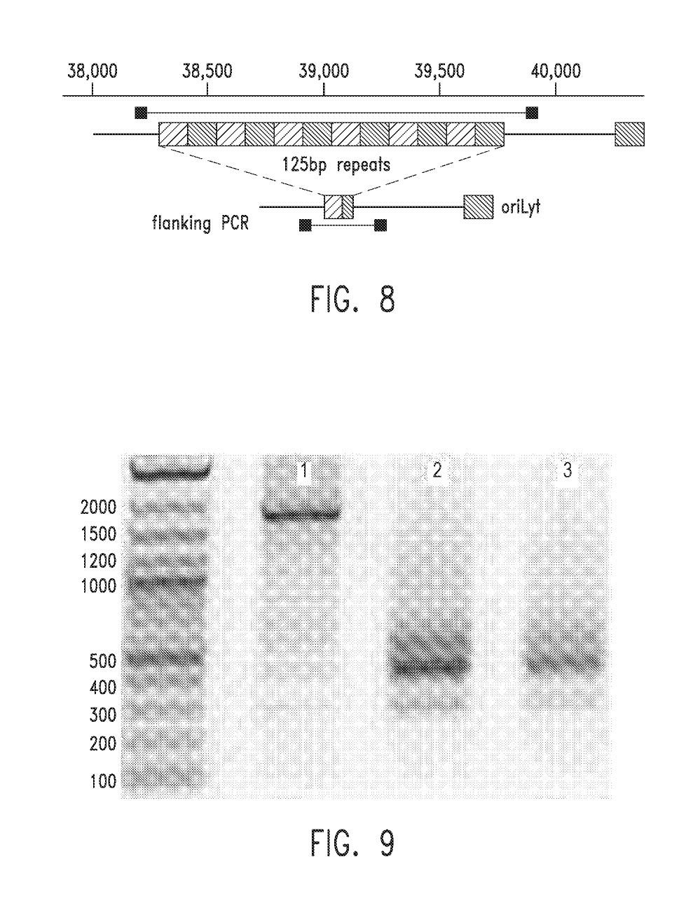

FIG. 8 shows genomic context around guide RNA sgEBV2 and PCR primer locations.

FIG. 9 shows a large deletion induced by sgEBV2, where lane 1-3 are before, 5 days after, and 7 days after sgEBV2 treatment, respectively. Guide RNA sgEBV2 targets a region with twelve 125-bp repeat units (FIG. 8). PCR amplicon of the whole repeat region gave a ˜1.8-kb band (FIG. 9). After 5 or 7 days of sgEBV2 transfection, we obtained ˜0.4-kb bands from the same PCR amplification (FIG. 9). The ˜1.4-kb deletion is the expected product of repair ligation between cuts in the first and the last repeat unit (FIG. 8).

DNA sequences flanking sgRNA targets were PCR amplified with Phusion DNA polymerase. SURVEYOR assays were performed following manufacturer's instruction. DNA amplicons with large deletions were TOPO cloned and single colonies were used for Sanger sequencing. EBV load was measured with Taqman digital PCR on Fluidigm BioMark. A Taqman assay targeting a conserved human locus was used for human DNA normalization. 1 ng of single-cell whole-genome amplification products from Fluidigm C1 were used for EBV quantitative PCR.

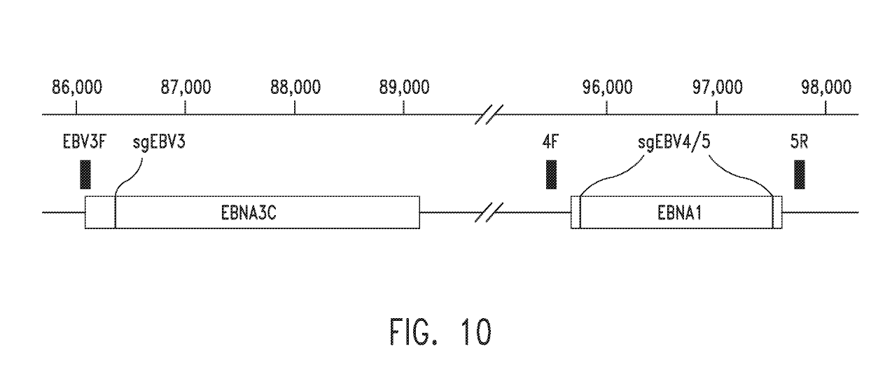

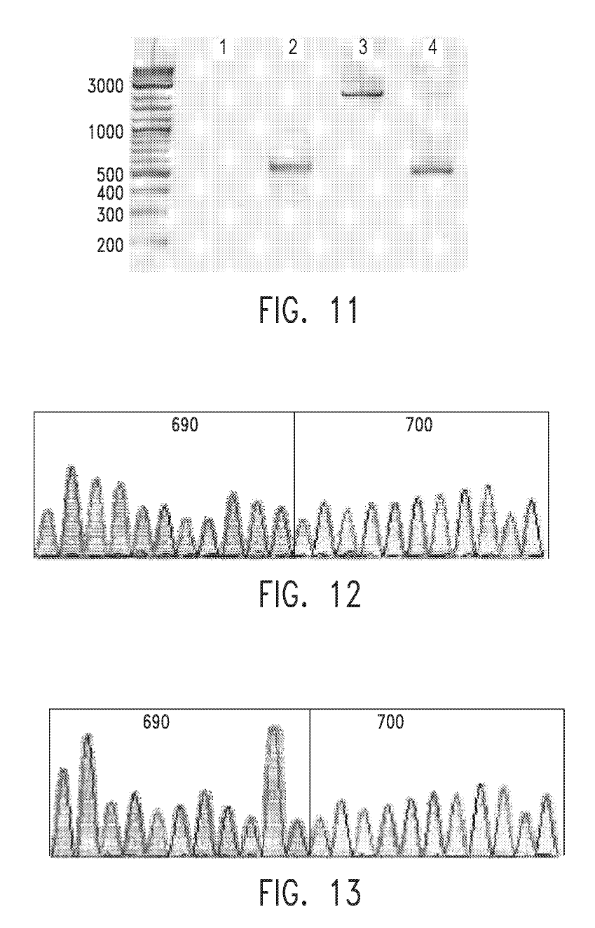

We further demonstrated that it is possible to delete regions between unique targets (FIG. 10). Six days after sgEBV4-5 transfection, PCR amplification of the whole flanking region (with primers EBV4F and 5R) returned a shorter amplicon, together with a much fainter band of the expected 2 kb (FIG. 11). Sanger sequencing of amplicon clones confirmed the direct connection of the two expected cutting sites (FIG. 13). A similar experiment with sgEBV3-5 also returned an even larger deletion, from EBNA3C to EBNA1 (FIG. 11-12).

Additional information such as primer design is shown in Wang and Quake, 2014, RNA-guided endonuclease provides a therapeutic strategy to cure latent herpesviridae infection, PNAS 111(36):13157-13162 and in the Supporting Information to that article published online at the PNAS website, and the contents of both of those documents are incorporated by reference for all purposes.

Cell Proliferation Arrest with EBV Genome Destruction.

Two days after CRISPR transfection, we flow sorted EGFP-positive cells for further culture and counted the live cells daily. FIG. 10 gives genome context around guide RNA sgEBV3/4/5 and PCR primer locations.

FIG. 11 shows large deletions induced by sgEBV3/5 and sgEBV4/5, where lane 1 and 2 are 3F/5R PCR amplicons before and 8 days after sgEBV3/5 treatment; and lane 3 and 4 are 4F/5R PCR amplicons before and 8 days after sgEBV4/5 treatment.

FIG. 12 shows that Sanger sequencing confirmed genome cleavage and repair ligation 8 days after sgEBV3/5 treatment.

FIG. 13 shows Sanger sequencing confirmed genome cleavage and repair ligation 8 days after sgEBV4/5 treatment.

FIG. 14 shows several cell proliferation curves after different CRISPR treatments.

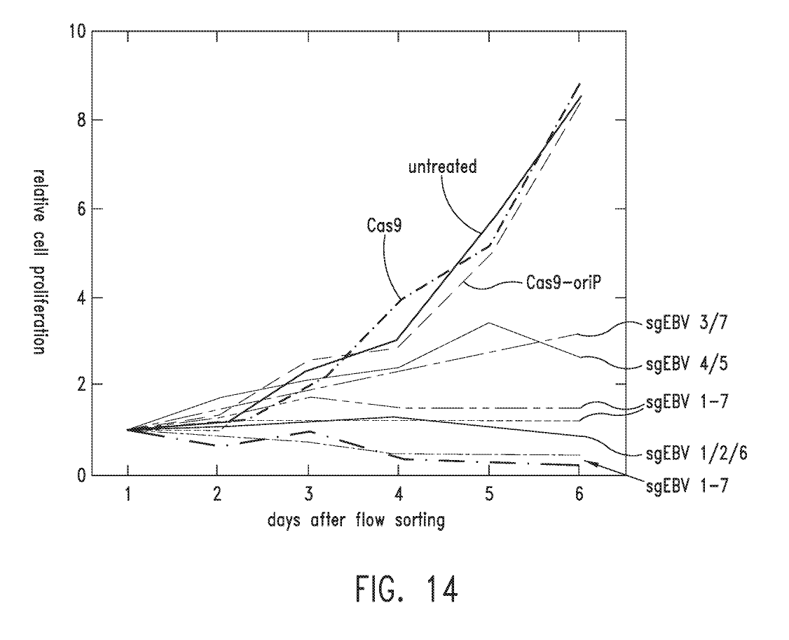

FIGS. 15-17 give flow cytometry scattering signals before (FIG. 15), 5 days after (FIG. 16) and 8 days after (FIG. 17) sgEBV1-7 treatments

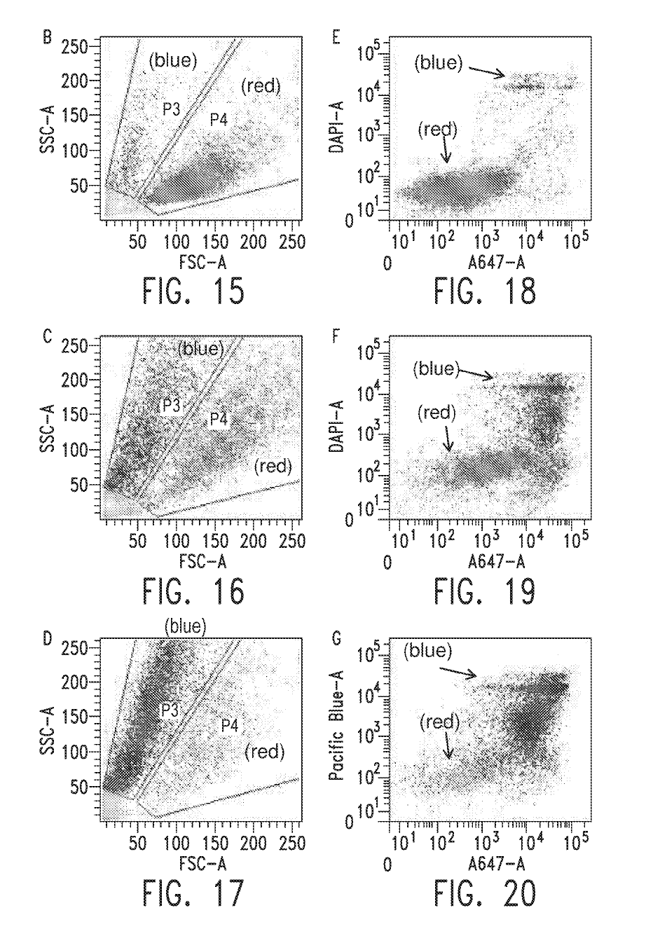

FIGS. 18-20 show Annexin V Alexa647 and DAPI staining results before (FIG. 18), 5 days after (FIG. 19) and 8 days after (FIG. 20) sgEBV1-7 treatments. Blue and red correspond to subpopulation P3 and P4 in (FIGS. 15-17).

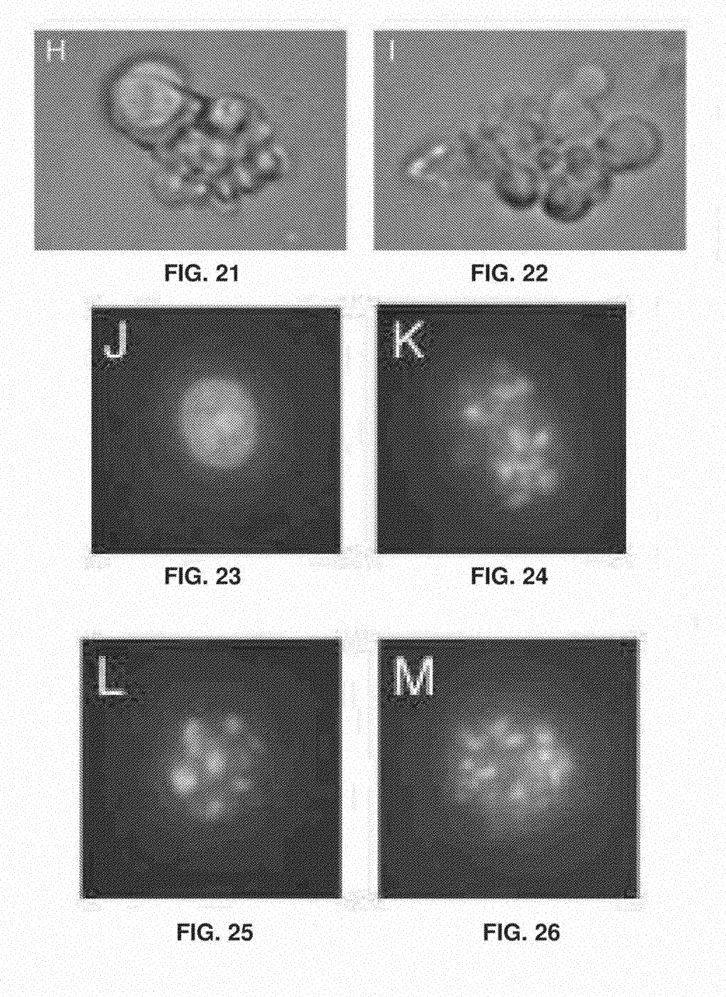

FIG. 21 shows that microscopy revealed apoptotic cell morphology after sgEBV1-7 treatment.

FIG. 22 shows that microscopy revealed apoptotic cell morphology after sgEBV1-7 treatment.

FIG. 23 shows nuclear morphology before sgEBV1-7 treatment.

FIG. 24 shows nuclear morphology after sgEBV1-7 treatment.

FIG. 25 shows nuclear morphology after sgEBV1-7 treatment.

FIG. 26 shows nuclear morphology after sgEBV1-7 treatment.

As expected, cells treated with Cas9 plasmids which lacked oriP or sgEBV lost EGFP expression within a few days and proliferated with a rate similar rate to the untreated control group (FIG. 14). Plasmids with Cas9-oriP and a scrambled guide RNA maintained EGFP expression after 8 days, but did not reduce the cell proliferation rate. Treatment with the mixed cocktail sgEBV1-7 resulted in no measurable cell proliferation and the total cell count either remained constant or decreased (FIG. 14). Flow cytometry scattering signals clearly revealed alterations in the cell morphology after sgEBV1-7 treatment, as the majority of the cells shrank in size with increasing granulation (FIGS. 15-17, population P4 to P3 shift). Cells in population P3 also demonstrated compromised membrane permeability by DAPI staining (FIGS. 18-20).

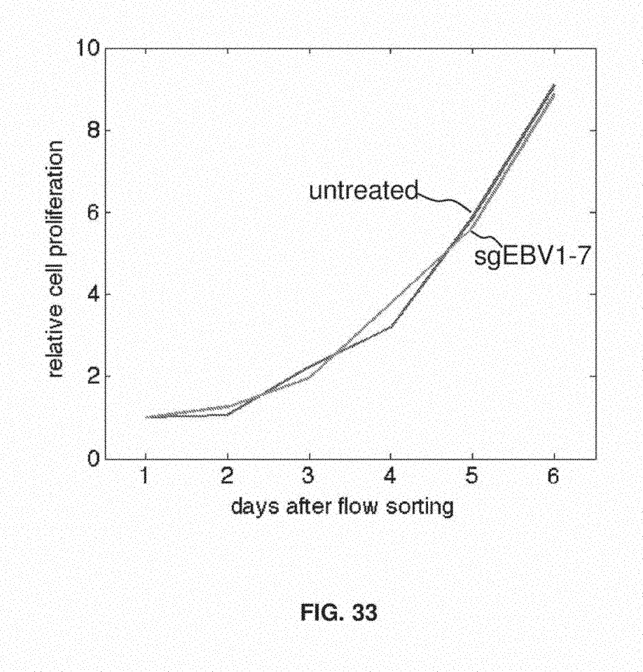

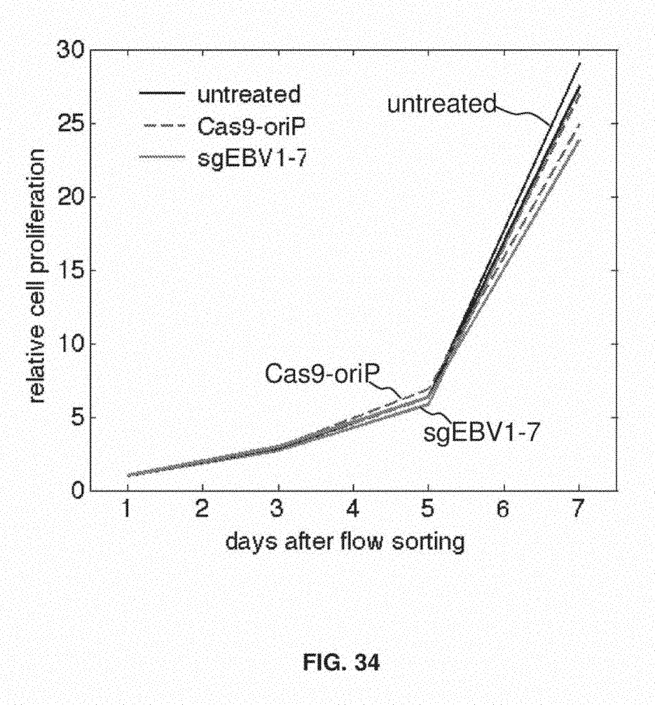

To rule out the possibility of CRISPR cytotoxicity, especially with multiple guide RNAs, we performed the same treatment on two other samples: the EBV-negative Burkitt's lymphoma cell line DG-75 (FIG. 33) and primary human lung fibroblast IMR90 (FIG. 34). FIG. 33 shows CRISPR cytotoxicity test with EBV-negative Burkitt's lymphoma DG-75.

FIG. 33 shows that the CRISPR treatments were not cytotoxic to the EBV-negative Burkitt's lymphoma cell line DG-75

FIG. 34 shows that the CRISPR treatments were not cytotoxic to primary human lung fibroblasts IMR90.

FIG. 34 represents CRISPR cytotoxicity test with primary human lung fibroblast IMR-90. Eight and nine days after transfection the cell proliferation rates did not change from the untreated control groups, suggesting neglectable cytotoxicity.

Previous studies have attributed the EBV tumorigenic ability to its interruption of host cell apoptosis (Ruf I K et al. (1999) Epstein-Barr Virus Regulates c-MYC, Apoptosis, and Tumorigenicity in Burkitt Lymphoma. Molecular and Cellular Biology 19:1651-1660). Suppressing EBV activities may therefore restore the apoptosis process, which could explain the cell death observed in our experiment. Annexin V staining revealed a distinct subpopulation of cells with intact cell membrane but exposed phosphatidylserine, suggesting cell death through apoptosis (FIGS. 18-20). Bright field microscopy showed obvious apoptotic cell morphology (FIGS. 21-22) and fluorescent staining demonstrated drastic DNA fragmentation (FIGS. 23-26). Altogether this evidence suggests restoration of the normal host cell apoptosis pathway after EBV genome destruction.

Complete Clearance of EBV in a Subpopulation.

To study the potential connection between cell proliferation arrest and EBV genome editing, we quantified the EBV load in different samples with digital PCR targeting EBNA1. Another Taqman assay targeting a conserved human somatic locus served as the internal control for human DNA normalization.

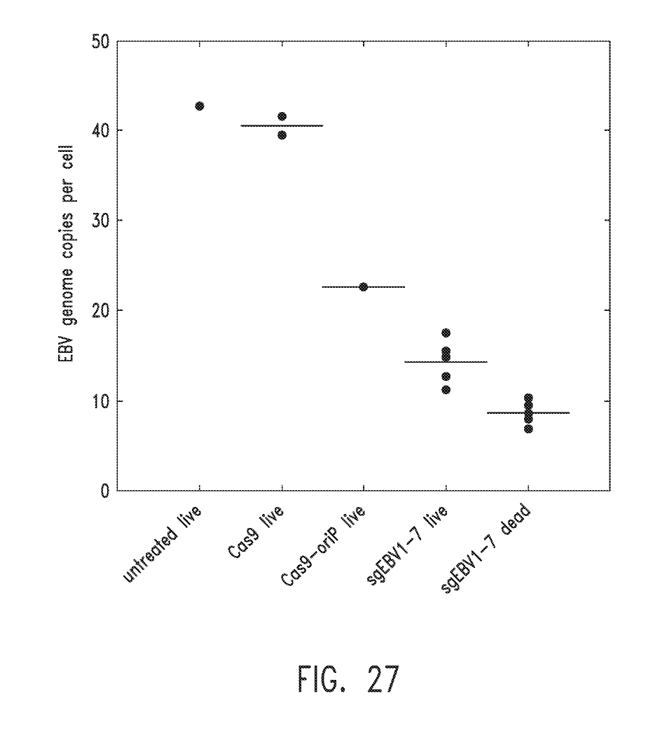

FIG. 27 shows EBV load after different CRISPR treatments by digital PCR, where Cas9 and Cas9-oriP had two replicates, and sgEBV1-7 had 5 replicates.

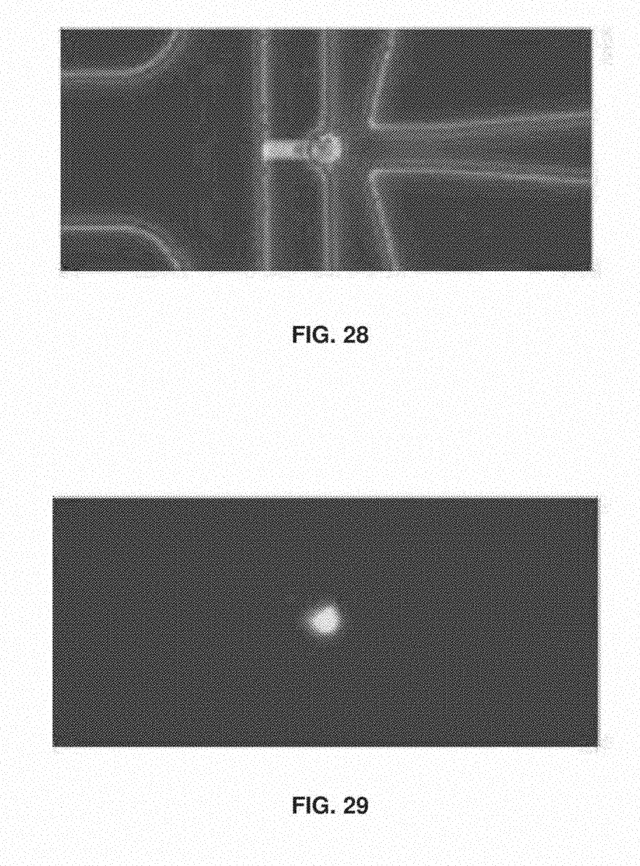

FIG. 28 shows microscopy of captured single cell for whole-genome amplification.

FIG. 29 shows microscopy of captured single cell for whole-genome amplification.

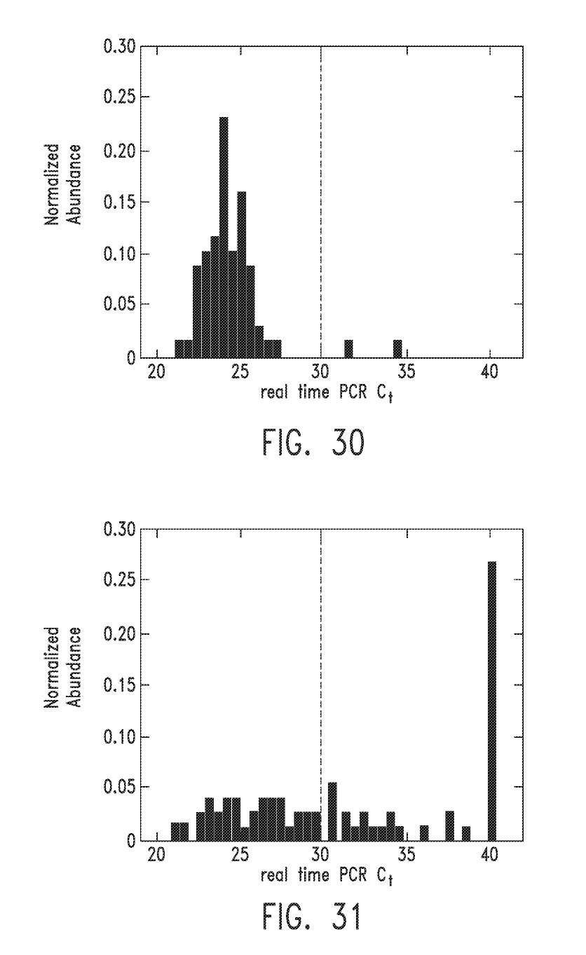

FIG. 30 gives a histogram of EBV quantitative PCR Ct values from single cells before treatment.

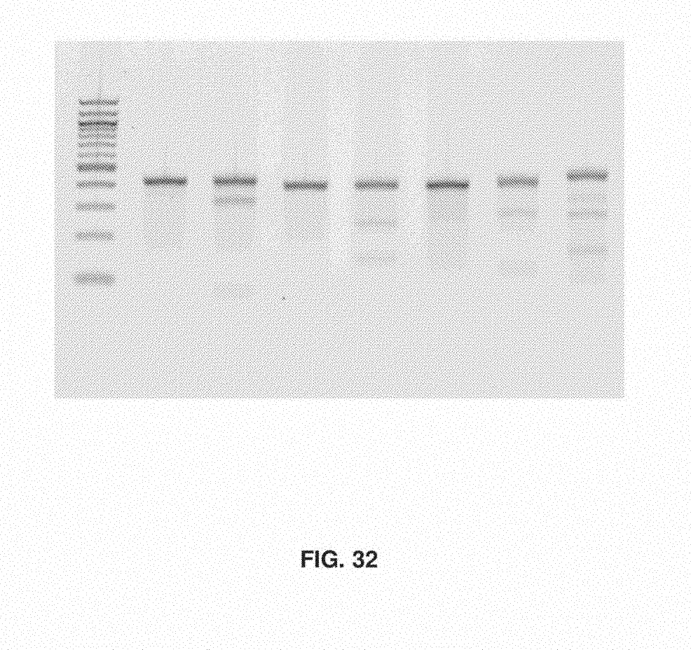

FIG. 31 gives a histogram of EBV quantitative PCR Ct values from single live cells 7 days after sgEBV1-7 treatment, where the dash lines in FIGS. 30 & 31 represent Ct values of one EBV genome per cell.

On average, each untreated Raji cell has 42 copies of EBV genome (FIG. 27). Cells treated with a Cas9 plasmid that lacked oriP or sgEBV did not have an obvious difference in EBV load difference from the untreated control. Cells treated with a Cas9-plasmid with oriP but no sgEBV had an EBV load that was reduced by ˜50%. In conjunction with the prior observation that cells from this experiment did not show any difference in proliferation rate, we interpret this as likely due to competition for EBNA1 binding during plasmid replication. The addition of the guide RNA cocktail sgEBV1-7 to the transfection dramatically reduced the EBV load. Both the live and dead cells have >60% EBV decrease comparing to the untreated control.

Although we provided seven guide RNAs at the same molar ratio, the plasmid transfection and replication process is likely quite stochastic. Some cells will inevitably receive different subsets or mixtures of the guide RNA cocktail, which might affect the treatment efficiency. To control for such effects, we measured EBV load at the single cell level by employing single-cell whole-genome amplification with an automated microfluidic system. We loaded freshly cultured Raji cells onto the microfluidic chip and captured 81 single cells (FIG. 28). For the sgEBV1-7 treated cells, we flow sorted the live cells eight days after transfection and captured 91 single cells (FIG. 29). Following manufacturer's instruction, we obtained ˜150 ng amplified DNA from each single cell reaction chamber. For quality control purposes we performed 4-loci human somatic DNA quantitative PCR on each single cell amplification product (Wang J, Fan H C, Behr B, Quake S R (2012) Genome-wide single-cell analysis of recombination activity and de novo mutation rates in human sperm. Cell 150:402-412) and required positive amplification from at least one locus. 69 untreated single-cell products passed the quality control and displayed a log-normal distribution of EBV load (FIG. 30) with almost every cell displaying significant amounts of EBV genomic DNA. We calibrated the quantitative PCR assay with a subclone of Namalwa Burkitt's lymphoma cells, which contain a single integrated EBV genome. The single-copy EBV measurements gave a Ct of 29.8, which enabled us to determine that the mean Ct of the 69 Raji single cell samples corresponded to 42 EBV copies per cells, in concordance with the bulk digital PCR measurement. For the sgEBV1-7 treated sample, 71 single-cell products passed the quality control and the EBV load distribution was dramatically wider (FIG. 31). While 22 cells had the same EBV load as the untreated cells, 19 cells had no detectable EBV and the remaining 30 cells displayed dramatic EBV load decrease from the untreated sample.

Essential Targets For EBV Treatment. The seven guide RNAs in our CRISPR cocktail target three different categories of sequences which are important for EBV genome structure, host cell transformation, and infection latency, respectively. To understand the most essential targets for effective EBV treatment, we transfected Raji cells with subsets of guide RNAs. Although sgEBV4/5 reduced the EBV genome by 85%, they could not suppress cell proliferation as effectively as the full cocktail (FIG. 14). Guide RNAs targeting the structural sequences (sgEBV1/2/6) could stop cell proliferation completely, despite not eliminating the full EBV load (26% decrease). Given the high efficiency of genome editing and the proliferation arrest, we suspect that the residual EBV genome signature in sgEBV1/2/6 was not due to intact genomes but to free-floating DNA that has been digested out of the EBV genome, i.e. as a false positive. We conclude that systematic destruction of EBV genome structure appears to be more effective than targeting specific key proteins for EBV treatment.

### Example 2: HPV Genome and Targets

The HPV genome is a double-stranded, circular DNA genome approximately 8 kb in size that can be divided, in general, into three major regions (early, late, and a long control region (LCR), which regions are separated by two polyadenylation sites. The early region is over 50% of the HPV genome from its 5′ half and encodes six common open reading frames (E1, E2, E4, E5, E6 and E7) that translate proteins. The late region is downstream of the early region and encodes L1 and L2 ORFs for translation of a major (L1) and a minor (L2) capsid protein. A targeting sequence such as a gRNA may be targeted to a capsid protein to interrupt viral function. The ˜850 bp LCR region has no protein-coding function, but bears the origin of replication as well as transcription factor binding sites for transcription regulation from viral early as well as late promoters. See Bernard, 2007, Gene expression of genital human papillomaviruses and considerations on potential antiviral approaches. Antivir. Ther. 7:219-237 incorporated by reference. The HPV-16 genome contains two major promoters. The P97 promoter lies upstream of the E6 ORF and is responsible for almost all early gene expression. The P670 promoter lies within the E7 ORF region and is responsible for late gene expression. The HPV-16 P97 promoter, equivalent to P99 in HPV-31 and P105 in HPV-18, is very potent and tightly controlled, primarily by upstream cis-elements in the LCR that interact with cellular transcription factors and the viral transactivator/repressor E2 and regulate the transcription of P97 from undifferentiated basal cells to highly differentiated keratinocytes. It is believed that E2 functions as a repressor for P97 transcription after TBP or TFIID binding and its transcriptional repression only occurs in cells harboring integrated, but not episomal HPV-16 DNA. The HPV-16 P670 promoter is a late-promoter and its activity can be induced only in differentiated keratinocytes. Elements in the E6 and E7 coding regions may regulate late promoters and both the late P670 promoter in HPV-16 and P742 in HPV-31 are positioned in the E7 coding region and transcription from the late promoter has to bypass the early pA site to allow expression of the late region. See Zheng & Baker, 2006, Papillomavirus genome structure, expression, and post-transcriptional regulation, Front Biosci 11:2286-2302, incorporated by reference.

The promoters may be used in a vector containing a gene for an antiviral, or targetable, endonuclease.

### Example 3

FIG. 36 shows a method 3201 for treating a cell 3237 to remove foreign nucleic acid such as a viral nucleic acid 3251. The method 3201 may be used clinically to treat an HPV infection, or the method 3201 may be used in vitro e.g., for research and development to remove foreign nucleic acid from subject cells such as cells from a human.

The method 3201 includes the steps of: forming 3225 a ribonucleoprotein (RNP) 3231 that includes a nuclease 3205 and an RNA 3213; delivering 3245 the RNP 3231 to infected cells 3237; and cleaving viral nucleic acid 3251 within the cells 3237 with the RNP 3231.

The delivering 3245 may include electroporation, or the RNP may be packaged in a liposome for the delivering 3245. In some embodiments, the viral nucleic acid 3251 will exist as an episomal viral genome, i.e., an episome or episomal vector, of a virus. The RNA 3213 has a portion that is substantially complementary to a target within a viral nucleic acid 3251 and preferably not substantially complementary to any location on a human genome. In the preferred embodiments, the virus is a Human Papilloma Virus (HPV) such as HPV-16.

In a preferred embodiment, the nuclease 3205 is a Crisper-associated protein such as, preferably, Cas9. The RNA 3213 may be a single guide RNA (sgRNA) (providing the functionality of crRNA and tracrRNA). In this preferred embodiment, the nuclease 3205 and the RNA 3213 are delivered to the cell as the RNP 3231.

In some embodiments, the RNP is delivered to tissue that is infected or suspected of being infected. For example, the RNP can be packed in liposomes and delivered topically or transdermally for clinical applications. Electroporation or nucleoporation may be used, which strategies may have particular value in ex vivo application.

In some embodiments, it may be found that RNP is preferable (e.g., to plasmid DNA) for clinical applications, particularly for parenteral delivery. RNP is the active pre-formed drug which offers advantages to DNA (AAV) or mRNA. No need to transcribe, translate, or assemble drug components within cell. Delivery of RNP 3231 may offer improved drug properties, e.g. earlier onset activity and controlled clearance (toxicity).

### Example 4: Cas9 RNP Kills HPV+ Cancer Cells

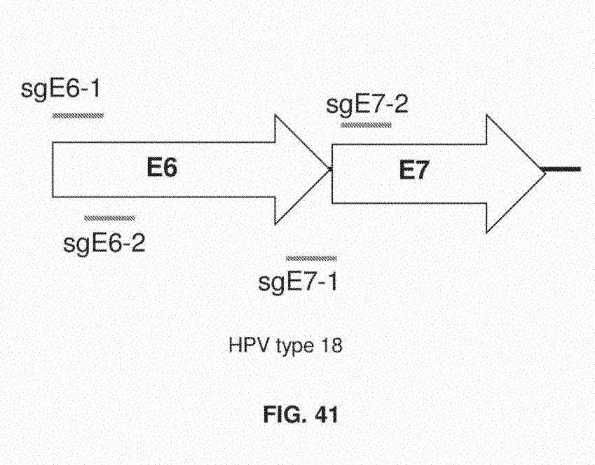

A nuclease such as Cas9 may be guided to an HPV genome through selected guide RNAs. FIG. 41 shows where the selected guide RNAs map to the HPV genome according to certain embodiments. Two map to the E6 gene and two map to the E7 gene.

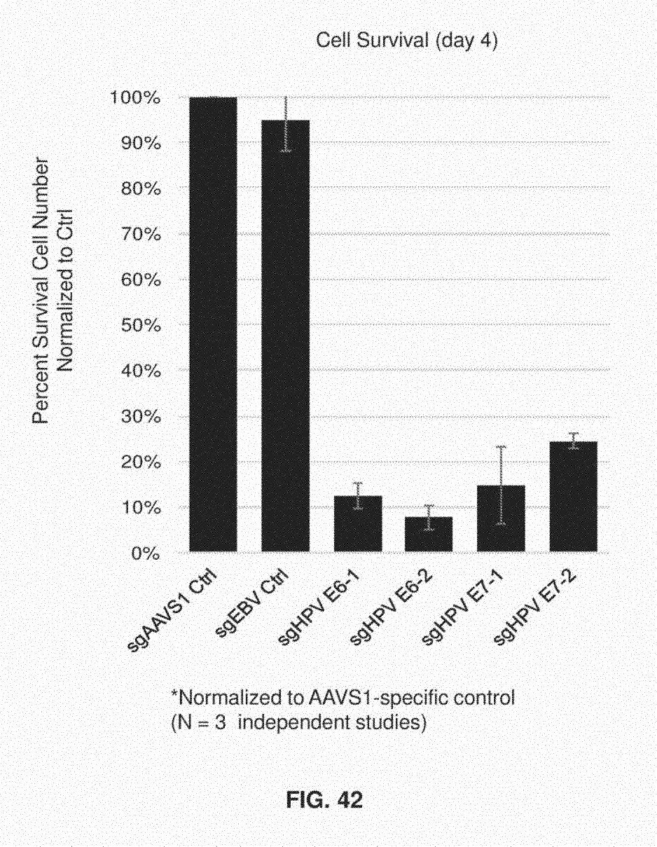

FIG. 42 shows the percent survival after treatment with Cas9 and each of sgHPV E6-1, sgHPV E6-2, sgHPV E7-1, and sgHPV E7-2. Treatment with HPV-Specific CRISPR/Cas9 ribonucleoprotein (RNP) kills HPV+ cancer cells.

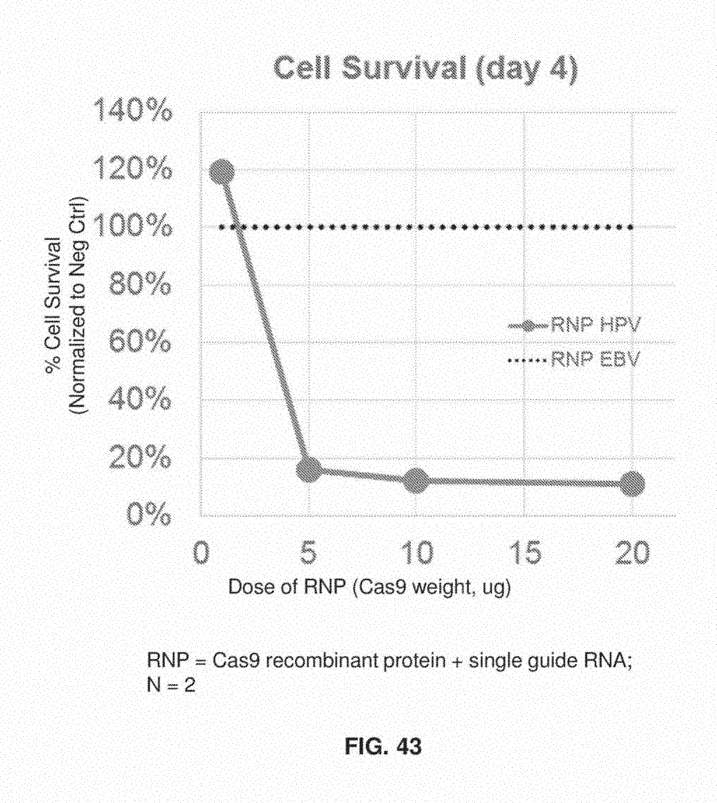

FIG. 43 shows the HPV-specific CRISPR/Cas9 RNP dose response in HPV+ cancer cells.

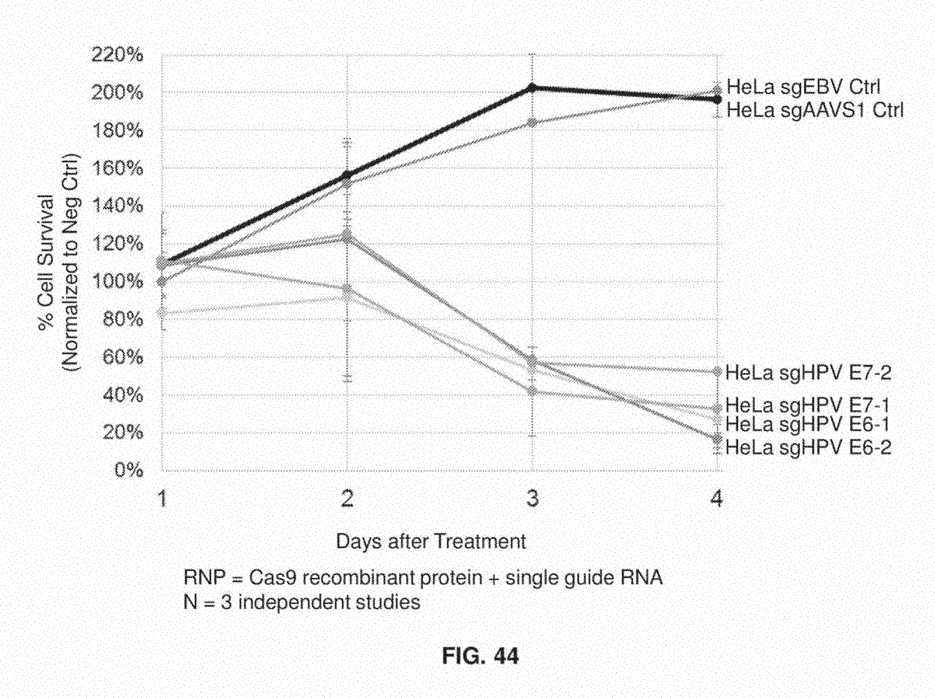

FIG. 44 gives the HPV-specific CRISPR/Cas9 RNP time-course in HPV+ cancer cells. FIGS. 42-44 demonstrate that delivering RNP to HPV+ cancer cells, wherein the RNP includes Cas9 and one or more guide RNAs mapping to the early genes, may kill the cancer cells.

### Example 5: Cas9 RNP Better Cleavage than Cas9 pDNA

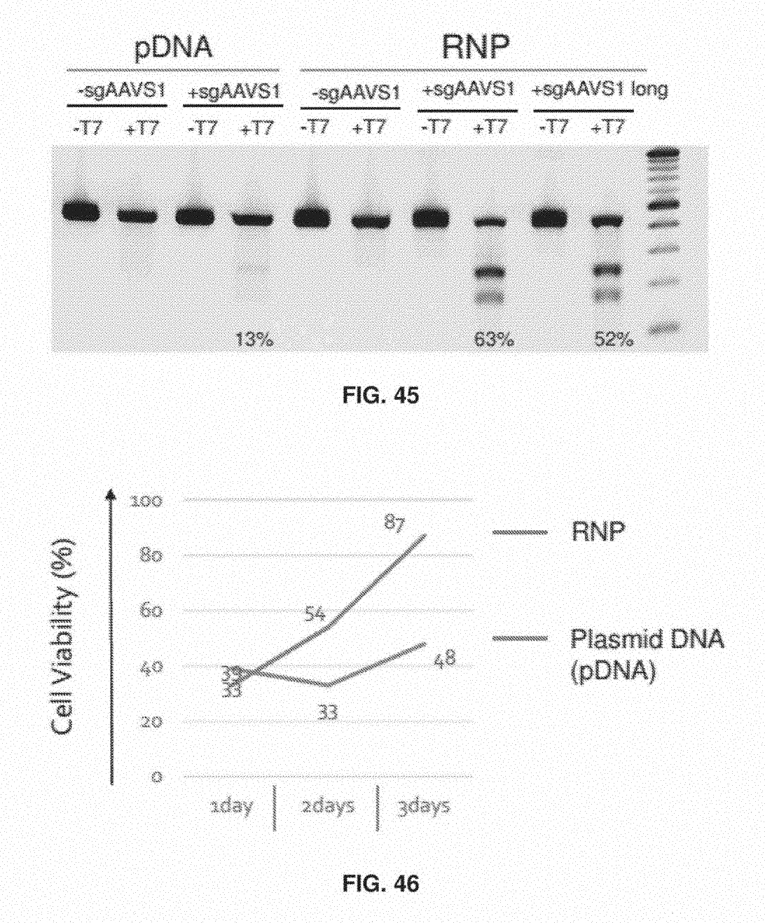

FIG. 45 is a gel showing that CRISPR/Cas9 RNP Enhances DNA cleavage.

FIG. 46 shows that RNP has decreased cytotoxicity relative to pDNA. Those results indicate that Cas9 in RNP form promise to be effective as viral treatment agents.

### Example 6: Cas9 RNP Kills HPV+ Cancer Cells More Effectively than pDNA

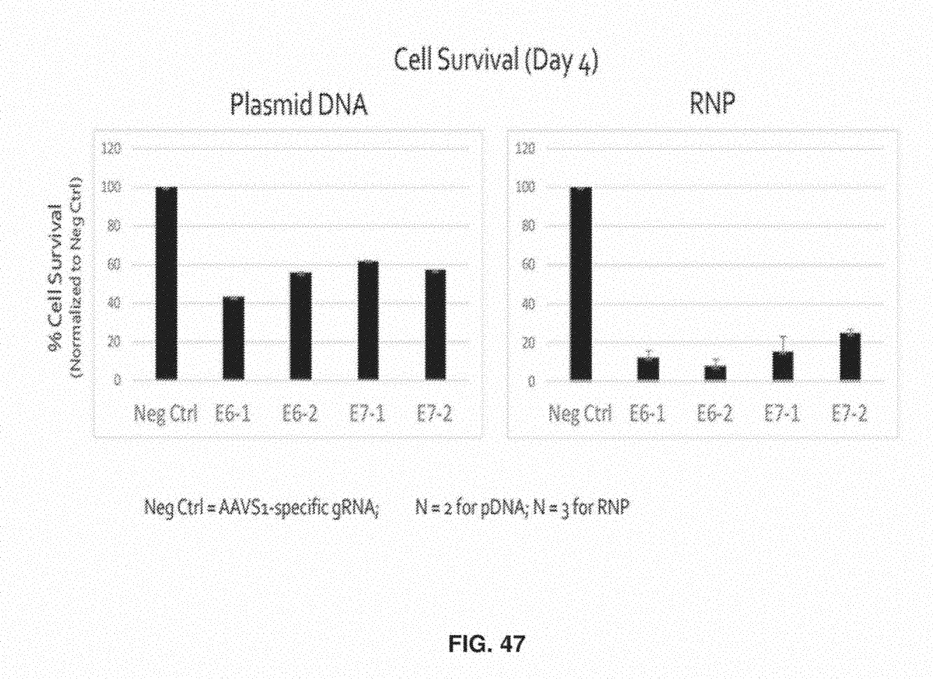

FIG. 47 shows that HPV+ cancer cell survival is lower when HPV-specific CRISPR/Cas9 RNP used to treat HPV+ cancer cells than when those cells are treated with pDNA encoding Cas9.

### Example 7: Multiple Guide RNAs Better than Individual Ones

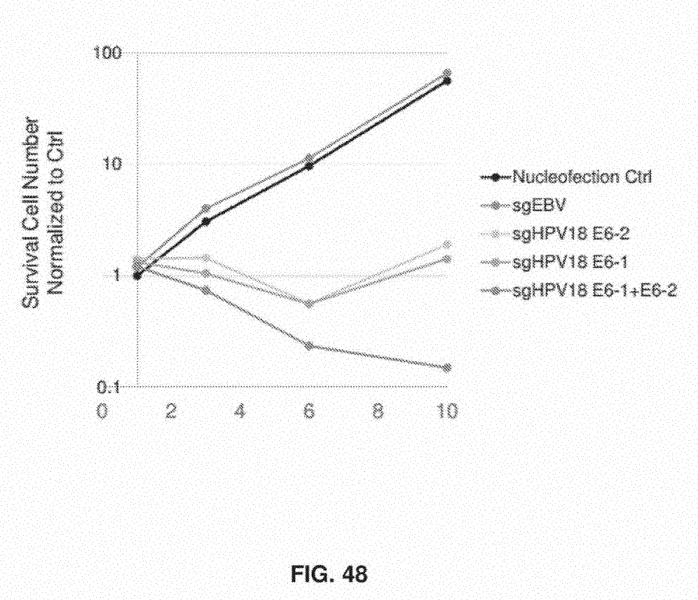

FIG. 48 shows that A Combination of HPV-Specific CRISPR RNPs improves HPV+ cancer cell killing. Delivering RNP with the E6-1 and E6-2 guide RNAs results in lower survival numbers than delivering RNP with only either one of those.

### Example 8: Killing HPV+ Cancer Cells by Targeting E6, E7

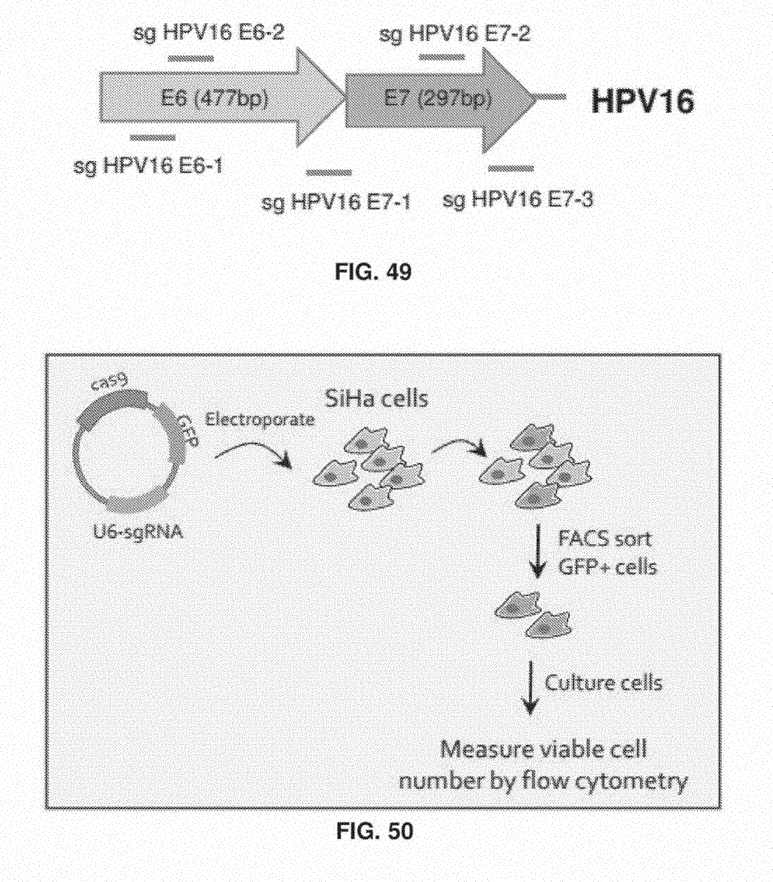

FIG. 49 shows the primer design for killing HPV+ cancer cells. The primers E6-1, E6-2, E7-1, E7-3, and E7-4 were designed to sit in the E6 and E7 genes as shown.

FIG. 50 shows a process for monitoring cell survival. A Cas9 plasmid with a GFP reporter is electroporated into cells. GFP+ cells are selected, cultured, and measured by flow cytometry.

FIG. 51 shows that HPV-16 specific CRISPR/Cas9 pDNA kills HPV-16 positive cancer cells. The normalized survival numbers show that the control cells had high survivorship. The cells not surviving were those treated with Cas9 and one of the E6-1, E7-2, and E7-3 primers.

### Example 9: Liposomal Delivery of RNP Inhibits Cancer Cells

FIG. 52 illustrated delivery of Cas9 RNP via liposome.

FIG. 53 shows that HPV-Specific CRISPR/Cas9 RNP formulated into a liposome inhibits HPV+ cancer cells. These results indicate that liposomal delivery of Cas9 RNP is promising an an antiviral/anticancer therapeutic, particularly for oncoviral cancer cells.

### Example 10: EBV-Specific CRISPR/Cas9 RNP Specifically Kills EBV+B Lymphoma Cancer Cells

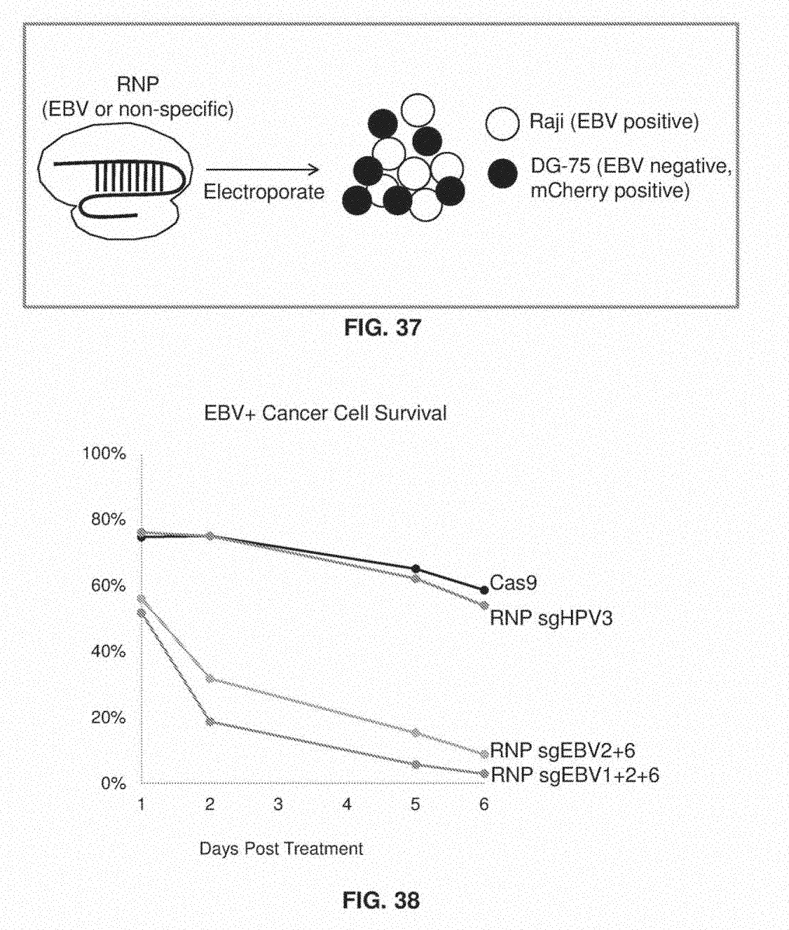

FIG. 37 diagrams an experimental design to show that EBV-specific CRISPR/Cas9 RNP specifically kills EBV+B lymphoma cancer cells. The Raji cells are EBV positive. Raji cells are a continuous human cell line of hematopoetic origin. The DG-75 cells are an EBV-negative B lymphocyte cell line available from American Type Culture Collection (Manassas, Va.). The DG-75 exhibits an mCherry fluorescent marker. Since the EBV negative cells contain a fluorescent marker, successful cleavage events can be identified.

FIG. 38 shows EBV+ cancer cell survival for 6 days post-treatment. Those EBV+ cells that received the RNP 3231 with guide RNAs substantially complementary to Epstein-Barr viral nucleic acid 3251 exhibited <10% survival rate, compared to about 60-70% in controls.

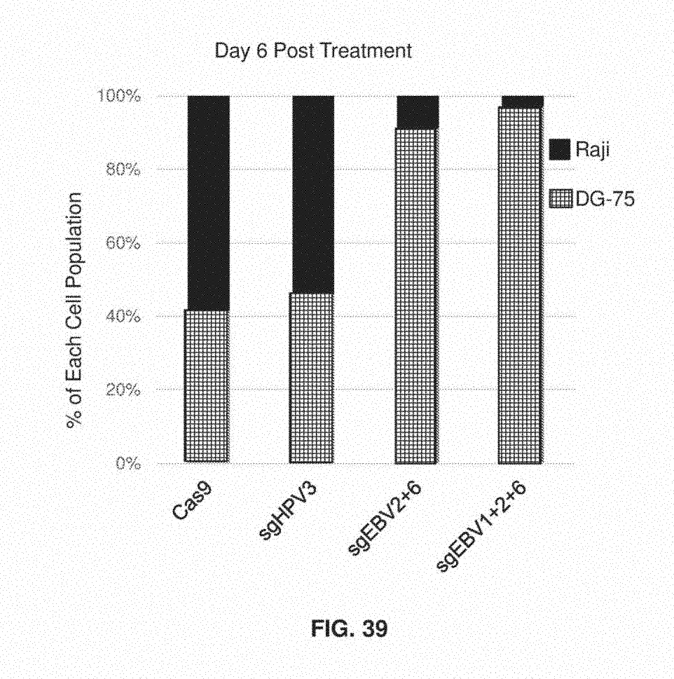

FIG. 39 shows the percent of each cell population at day 6 post-treatment for Cas9, sgHPV3, sgEBV2+6, and sgEBV1+2+6. This snapshot at day 6 shows that the DG-75 treated with the RNP 3231 with guide RNAs substantially complementary to Epstein-Barr viral nucleic acid 3251 dominated the cultures over the Raji cells.

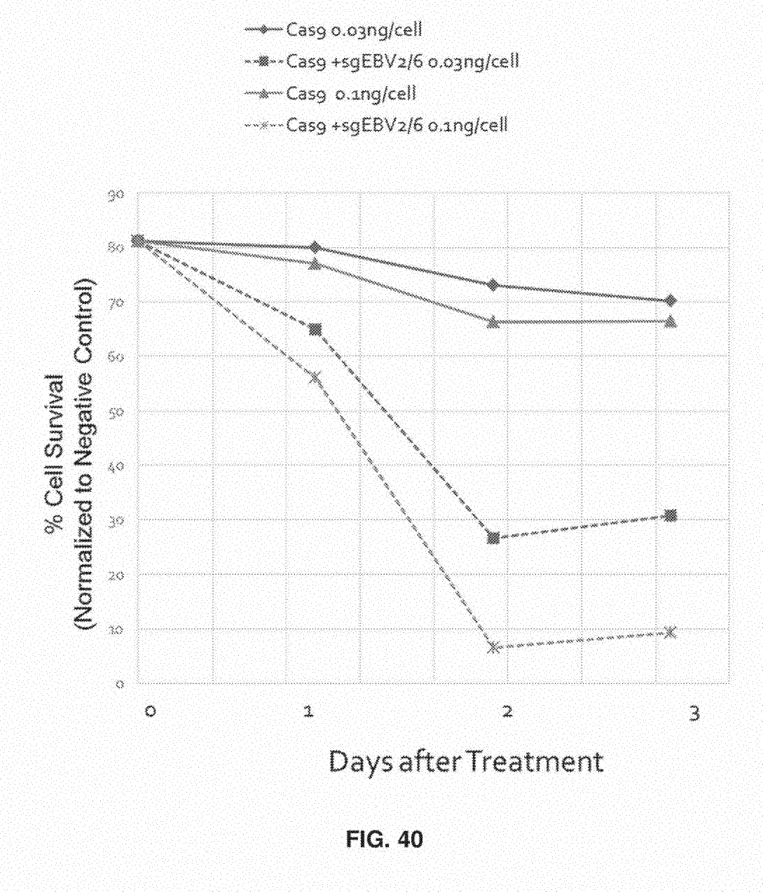

FIG. 40 shows the percent cell survivial (normalized to a negative control) for 3 days after treatment for Cas9 (at 0.03 & 0.1 ng/cell) as well as for Cas9 with sgEBV2/6 (at the same doses).

1.-21. (canceled)

22. A composition comprising a ribonucleoprotein (RNP), wherein the RNP comprises a Cas9 nuclease and a guide RNA complementary to a target site within a viral nucleic acid of a human papillomavirus (HPV).

23. The composition of claim 22, further comprising a liposome enveloping the RNP.

24. The composition of claim 22, wherein the target site lies within an E6 or E7 gene.

25. The composition of claim 24, wherein the HPV is HPV-16.

26. The composition of claim 24, wherein the HPV is HPV-18.

27. The composition of claim 22, wherein the Cas9 nuclease includes at least one nuclear localization signal.

28. The composition of claim 22, wherein the guide RNA is a single guide RNA (sgRNA).

29. The composition of claim 22, further comprising a second RNP, wherein the second RNP comprises a second Cas9 nuclease and a second guide RNA complementary to a different target site within the viral nucleic acid of the HPV.

30. The composition of claim 29, further comprising a second liposome enveloping the second RNP.

31. A pharmaceutical formulation comprising the composition of claim 22 and a pharmaceutically acceptable diluent, adjuvant, or carrier.

32. The pharmaceutical formulation of claim 31, wherein the pharmaceutical formulation is formulated for topical administration.

33. The pharmaceutical formulation of claim 32, wherein the RNP is enveloped within a liposome.

34. A method of treating an HPV infection in a subject in need thereof, comprising administering the composition of claim 22 to the subject.

35. The method of claim 34, wherein the composition further comprises a liposome enveloping the RNP.

36. The method of claim 34, wherein the target site lies within an E6 or E7 gene.

37. The method of claim 36, wherein the HPV is HPV-16.

38. The method of claim 36, wherein the HPV is HPV-18.

39. The method of claim 34, wherein the composition further comprises a second RNP, wherein the second RNP comprises a second Cas9 nuclease and a second guide RNA complementary to a different target site within the viral nucleic acid of the HPV.

40. A method of treating an HPV infection in a subject in need thereof, comprising topically administering the pharmaceutical composition of claim 32 to the subject.
